SEQUENCE LISTING (39 sequences)

SEQ ID NO: 1 - protein, 67 aa, synthetic construct
  REGION 1..67: note = Synthetic
MSVVNAKQTQIMGGGGRDEDNTEDSAQASGQVQINTEGVDSLLDEIDGLLENNAEEFVRSYVQKGGE

SEQ ID NO: 2 - protein, 28 aa, synthetic construct
  REGION 1..28: note = Synthetic
DSLLDEIDGLLENNAEEFVRSYVQKGGE

SEQ ID NO: 3 - protein, 63 aa, synthetic construct
  REGION 1..63: note = Synthetic
MTNPQSQISGGGDRPEDTNDDAQGLGQAQVNTAGTDDLLDEIDGLLEENAEEFVSSYVQKGGQ

SEQ ID NO: 4 - protein, 67 aa, synthetic construct
  REGION 1..67: note = Synthetic
MTTGGSGQGQVHGGRGRGDGPASGDVTASGQEQLKVSGTDDLLDEIDGLLESNAEEFVKSYVQKGGQ

SEQ ID NO: 5 - protein, 57 aa, synthetic construct
  REGION 1..57: note = Synthetic
MNAGGPNADDDSLDHSLGTAQAQISATGVDDLLDEIDGLLENNAEEFVRSYVQKGGQ

SEQ ID NO: 6 - protein, 60 aa, synthetic construct
  REGION 1..60: note = Synthetic
MAQQQIHGGSGNGSEDEGAFEAGQAQLNTSGTDDLLDEIDALLDNNAEEFVRSYVQKGGE

SEQ ID NO: 7 - protein, 62 aa, synthetic construct
  REGION 1..62: note = Synthetic
MSNQQQIHGHTGGGDDAEGTPAQAGQAQINTAGTDDLLDEIDALLDTNAEEFVRSFVQKGGQ

SEQ ID NO: 8 - protein, 63 aa, synthetic construct
  REGION 1..63: note = Synthetic
MSNKQSQVQGSGSGDNSDDDDVQAAGQVQINTTGTDDLLDEIDGLLESNAEEYVSSYVQKGGQ

SEQ ID NO: 9 - protein, 62 aa, synthetic construct
  REGION 1..62: note = Synthetic
MADKQVYSSGGKGPTDDDVVDGGAGQVQINTHEADSLLDEIDSLLETDSEEFVKSYVQKGGQ

SEQ ID NO: 10 - protein, 63 aa, synthetic construct
  REGION 1..63: note = Synthetic
MAQDQINISGGGDNGEGEPGDARNAGQVNVNTTGTDDLLDEIDALLDTNAEEFVRSYVQKGGQ

SEQ ID NO: 11 - protein, 919 aa, synthetic construct
  REGION 1..919: note = Synthetic
MAKKNKMKPRELREAQKKARQLKAAEINNNAAPAIAAMPAAEVIAPAAEKKKSSVKAAGMKSILVSENKMYITSFGKGNSAVLEYEVDNNDYNQTQLSSKDNSNIQLGGVNEVNITFSSKHGFESGVEINTSNPTHRSGESSPVRGDMLGLKSELEKRFFGKTFDDNIHIQLIYNILDIEKILAVYVTNIVYALNNMLGVKGSESHDDFIGYLSTNNIYDVFIDPDNSSLSDDKKANVRKSLSKFNALLKTKRLGYFGLEEPKTKDNRVSQAYKKRVYHMLAIVGQIRQCVFHDKSGAKRFDLYSFINNIDPEYRDTLDYLVEERLKSINKDFIEDNKVNISLLIDMMKGYEADDIIRLYYDFIVLKSQKNLGFSIKKLREKMLDEYGFRFKDKQYDSVRSKMYKLMDFLLFCNYYRNDIAAGESLVRKLRFSMTDDEKEGIYADEAAKLWGKFRNDFENIADHMNGDVIKELGKADMDFDEKILDSEKKNASDLLYFSKMIYMLTYFLDGKEINDLLTTLISKFDNIKEFLKIMKSSAVDVECELTAGYKLFNDSQRITNELFIVKNIASMRKPAASAKLTMFRDALTILGIDDKITDDRISGILKLKEKGKGIHGLRNFITNNVIESSRFVYLIKYANAQKIREVAKNEKVVMFVLGGIPDTQIERYYKSCVEFPDMNSSLGVKRSELARMIKNISFDDFKNVKQQAKGRENVAKERAKAVIGLYLTVMYLLVKNLVNVNARYVIAIHCLERDFGLYKEIIPELASKNLKNDYRILSQTLCELCDKSPNLFLKKNERLRKCVEVDINNADSSMTRKYRNCIAHLTVVRELKEYIGDICTVDSYFSIYHYVMQRCITKRENDTKQEEKIKYEDDLLKNHGYTKDFVKALNSPFGYNIPRFKNLSIEQLFDRNEYLTEK

SEQ ID NO: 12 - protein, 933 aa, synthetic construct
  REGION 1..933: note = Synthetic
MKKKMSLREKREAEKQAKKAAYSAASKNTDSKPAEKKAETPKPAEIISDNSRNKTAVKAAGLKSTIISGDKLYMTSFGKGNAAVIEQKIDINDYSFSAMKDTPSLEVDKAESKEISFSSHHPFVKNDKLTTYNPLYGGKDNPEKPVGRDMLGLKDKLEERYFGCTFNDNLHIQIIYNILDIEKILAVHSANITTALDHMVDEDDEKYLNSDYIGYMNTINTYDVFMDPSKNSSLSPKDRKNIDNSRAKFEKLLSTKRLGYFGFDYDANGKDKKKNEEIKKRLYHLTAFAGQLRQWSFHSAGNYPRTWLYKLDSLDKEYLDTLDHYFDKRFNDINDDFVTKNATNLYILKEVFPEANFKDIADLYYDFIVIKSHKNMGFSIKKLREKMLECDGADRIKEQDMDSVRSKLYKLIDFCIFKYYHEFPELSEKNVDILRAAVSDTKKDNLYSDEAARLWSIFKEKFLGFCDKIVVWVTGEHEKDITSVIDKDAYRNRSNVSYFSKLMYAMCFFLDGKEINDLLTTLINKFDNIANQIKTAKELGINTAFVKNYDFFNHSEKYVDELNIVKNIARMKKPSSNAKKAMYHDALTILGIPEDMDEKALDEELDLILEKKTDPVTGKPLKGKNPLRNFIANNVIENSRFIYLIKFCNPENVRKIVNNTKVTEFVLKRIPDAQIERYYKSCTDSEMNPPTEKKITELAGKLKDMNFGNFRNVRQSAKENMEKERFKAVIGLYLTVVYRVVKNLVDVNSRYIMAFHSLERDSQLYNVSVDNDYLALTDTLVKEGDNSRSRYLAGNKRLRDCVKQDIDNAKKWFVSDKYNSITKYRNNVAHLTAVRNCAEFIGDITKIDSYFALYHYLIQRQLAKGLDHERSGFDRNYPQYAPLFKWHTYVKDVVKALNAPFGYNIPRFKNLSIDALFDRNEIKKNDGEKKSDD

SEQ ID NO: 13 - protein, 944 aa, synthetic construct
  REGION 1..944: note = Synthetic
MAKKSKGMSLREKRELEKQKRIQKAAVNSVNDTPEKTEEANVVSVNVRTSAENKHSKKSAAKALGLKSGLVIGDELYLTSFGRGNEAKLEKKISGDTVEKLGIGAFEVAERDESTLTLESGRIKDKTARPKDPRHITVDTQGKFKEDMLGIRSVLEKKIFGKTFDDNIHVQLAYNILDVEKIMAQYVSDIVYMLHNTDKTERNDNLMGYMSIRNTYKTFCDTSNLPDDTKQKVENQKREFDKIIKSGRLGYFGEAFMVNSGNSTKLRPEKEIYHIFALMASLRQSYFHGYVKDTDYQGTTWAYTLEDKLKGPSHEFRETIDKIFDEGFSKISKDFGKMNKVNLQILEQMIGELYGSIERQNLTCDYYDFIQLKKHKYLGFSIKRLRETMLETTPAECYKAECYNSERQKLYKLIDFLIYDLYYNRKPARIEEIVDKLRESVNDEEKESIYSVEAKYVYESLSKVLDKSLKNSVSGETIKDLQKRYDDETANRIWDISQHSISGNVNCFCKLIYIMTLMLDGKEINDLLTTLVNKFDNIASFIDVMDELGLEHSFTDNYKMFADSKAICLDLQFINSFARMSKIDDEKSKRQLFRDALVILDIGNKDETWINNYLDSDIFKLDKEGNKLKGARHDFRNFIANNVIKSSRFKYLVKYSSADGMIKLKTNEKLIGFVLDKLPETQIDRYYESCGLDNAVVDKKVRIEKLSGLIRDMKFDDFSGVKTSNKAGDNDKQDKAKYQAIISLYLMVLYQIVKNMIYVNSRYVIAFHCLERDFGMYGKDFGKYYQGCRKLTDHFIEEKYMKEGKLGCNKKVGRYLKNNISCCTDGLINTYRNQVDHFAVVRKIGNYAAYIKSIGSWFELYHYVIQRIVFDEYRFALNNTESNYKNSIIKHHTYCKDMVKALNTPFGYDLPRYKNLSIGDLFDRNNYLNKTKESIDANSSIDSQ

SEQ ID NO: 14 - protein, 946 aa, synthetic construct
  REGION 1..946: note = Synthetic
MNNKRKTKAKAAGLKSVFFDQKQAVLTTFAKGNNSQIEKKVVNSEVKDLRQPPAFDLELKEKTFYISGKNNINTSRENPLASASLPLSKRQRIRAERIKRAREENRPYHNVKRVGEDDLRAKADLEKHYFGKEYSDNLKIQIIYNILDINKIISPYINDIVYSMNNLARNDEYIDGKIDVIGSLSSTTDYSSFMSPNKDLEKEKKFSFHRENYKKFVEASKPYMRYYGKVFIRDVKKSKLSTGKGEKIEVMYRSDEEIFTIFQILSYVRQSIMHNDIGNKSSILAIEKYPARFVGFLSDLLKTKTNDVNRMFIDNNSQTNFWVLFSIFGLQDHTSGADKICRNFYDFVIKADSKNLGFSLKKIRELMLDLPNANMLRDHQFDTVRSKFYTLLDFIIYQHYLEEKSRIDNMVEKLRMTLKEEEKEVLYAAEAKIVWNAIGAKVINKLVPMMNGDALKEIKRKNRDRKLPQSVIATVQVNSDANVFSGLIYFLTLFLDGKEINEMVSNLITKFENIDSLLHVDREIYKSDEKDLDLEIEKLALFFKGVVRPNAKTDTGAGEISKSFSIFQSAERIIEELKFIKNVTRMDNEIFPSEGVFLDAANVLGVRGDDFDFSNEFVGDDLHSDANKKIINKINGTKEDRNLRNFIINNVVKSRRFQYIARHMNTHYVKQLANNETLNRFVLNKMGDAKIINRYYESISGNTPNIEVRSQIDYLVKRLRSFSFEDLNDVKQKVRPGTNESIEKEKKKALVGLCLTIQYLVYKNLVNINARYTTAFYCLERDSKLKGFGVDVWRDFESYTALTNHFIKEGYLPVRKAEILRANLKHLDCEDGFKYYRNQVTHLNAIRVAYKYINEIKSVHSYFALYHYIMQRHLYDSLQAKAKDSSGFVIDALKKSFEHKIYSKDLLHVLHSPFGYNTARYKNLSIEALFDKNESRPEVNPLSTND

SEQ ID NO: 15 - protein, 906 aa, synthetic construct
  REGION 1..906: note = Synthetic
MEREVKKPPKKSLAKAAGLKSTFVISPQEKELAMTAFGRGNDALLQKRIVDGVVRDVAGEKQQFQVQRQDESRFRLQNSRLADRTVTADDPLHRAETPRRQPLGAGMDQLRRKAILEQKYFGRTFDDNIHIQLIYNILDIHKMLAVPANHIVHTLNLLGGYGETDFVGMLPAGLPYDKLRVVKKKNGDTVDIKADIAAYAKRPQLAYLGAAFYDVTPGKSKRDAARGRVKREQDVYAILSLMSLLRQFCAHDSVRIWGQNTTAALYHLQALPQDMKDLLDDGWRRALGGVNDHFLDTNKVNLLTLFEYYGAETKQARVALTQDFYRFVVLKEQKNMGFSLRRLREELLKLPDAAYLTGQEYDSVRQKLYMLLDFLLCRLYAQERADRCEELVSALRCALSDEEKDTVYQAEAAALWQALGDTLRRKLLPLLKGKKLQDKDKKKSDELGLSRDVLDGVLFRPAQQGSRANADYFCRLMHLSTWFMDGKEINTLLTTLISKLENIDSLRSVLESMGLAYSFVPAYAMFDHSRYIAGQLRVVNNIARMRKPAIGAKREMYRAAVVLLGVDSPEAAAAITDDLLQIDPETGKVRPRSDSARDTGLRNFIANNVVESRRFTYLLRYMTPEQARVLAQNEKLIAFVLSTVPDTQLERYCRTCGREDITGRPAQIRYLTAQIMGVRYESFTDVEQRGRGDNPKKERYKALIGLYLTVLYLAVKNMVNCNARYVIAFYCRDRDTALYQKEVCWYDLEEDKKSGKQRQVEDYTALTRYFVSQGYLNRHACGYLRSNMNGISNSLLTAYRNAVDHLNAIPPLGSLCRDIGRVDSYFALYHYAVQQYLNGRYYRKTPREQELFAAMAQHRTWCSDLVKALNTPFGYNLARYKNLSIDGLFDREGDHVVREDGEKPAE

SEQ ID NO: 16 - protein, 954 aa, synthetic construct
  REGION 1..954: note = Synthetic
MGKKIHARDLREQRKTDRTEKFADQNKKREAERAVPKKDAAVSVKSVSSVSSKKDNVTKSMAKAAGVKSVFAVGNTVYMTSFGRGNDAVLEQKIVDTSHEPLNIDDPAYQLNVVTMNGYSVTGHRGETVSAVTDNPLRRFNGRKKDEPEQSVPTDMLCLKPTLEKKFFGKEFDDNIHIQLIYNILDIEKILAVYSTNAIYALNNMSADENIENSDFFMKRTTDETFDDFEKKKESTNSREKADFDAFEKFIGNYRLAYFADAFYVNKKNPKGKAKNVLREDKELYSVLTLIGKLRHWCVHSEEGRAEFWLYKLDELKDDFKNVLDVVYNRPVEEINNRFIENNKVNIQILGSVYKNTDIAELVRSYYEFLITKKYKNMGFSIKKLRESMLEGKGYADKEYDSVRNKLYQMTDFILYTGYINEDSDRADDLVNTLRSSLKEDDKTTVYCKEADYLWKKYRESIREVADALDGDNIKKLSKSNIEIQEDKLRKCFISYADSVSEFTKLIYLLTRFLSGKEINDLVTTLINKFDNIRSFLEIMDELGLDRTFTAEYSFFEGSTKYLAELVELNSFVKSCSFDINAKRTMYRDALDILGIESDKTEEDIEKMIDNILQIDANGDKKLKKNNGLRNFIASNVIDSNRFKYLVRYGNPKKIRETAKCKPAVRFVLNEIPDAQIERYYEACCPKNTALCSANKRREKLADMIAEIKFENFSDAGNYQKANVTSRTSEAEIKRKNQAIIRLYLTVMYIMLKNLVNVNARYVIAFHCVERDTKLYAESGLEVGNIEKNKTNLTMAVMGVKLENGIIKTEFDKSFAENAANRYLRNARWYKLILDNLKKSERAVVNEFRNTVCHLNAIRNININIKEIKEVENYFALYHYLIQKHLENRFADKKVERDTGDFISKLEEHKTYCKDFVKAYCTPFGYNLVRYKNLTIDGLFDKNYPGKDDSDEQK

SEQ ID NO: 17 - protein, 967 aa, synthetic construct
  REGION 1..967: note = Synthetic
MIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLRHWVVHNNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFRNKAVHLEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNS

SEQ ID NO: 18 - protein, 7 aa, synthetic construct
  REGION 1..7: note = Synthetic
PKKKRKV

SEQ ID NO: 19 - protein, 16 aa, synthetic construct
  REGION 1..16: note = Synthetic
KRPAATKKAGQAKKKK

SEQ ID NO: 20 - protein, 20 aa, synthetic construct
  REGION 1..20: note = Synthetic
AVKRPAATKKAGQAKKKKLD

SEQ ID NO: 21 - protein, 25 aa, synthetic construct
  REGION 1..25: note = Synthetic
MSRRRKANPTKLSENAKKLAKEVEN

SEQ ID NO: 22 - protein, 9 aa, synthetic construct
  REGION 1..9: note = Synthetic
PAAKRVKLD

SEQ ID NO: 23 - protein, 9 aa, synthetic construct
  REGION 1..9: note = Synthetic
KLKIKRPVK

SEQ ID NO: 24 - protein, 482 aa, synthetic construct
  REGION 1..482: note = Synthetic
MSTVESALTRRIMGIETEYGLTFVDGDSKKLRPDEIARRMFRPIVEKYSSSNIFIPNGSRLYLDVGSHPEYATAECDNLTQLINFEKAGDVIADRMAVDAEESLAKEDIAGQVYLFKNNVDSVGNSYGCHENYLVGRSMPLKALGKRLMPFLITRQLICGAGRIHHPNPLDKGESFPLGYCVSQRSDHVWEGVSSATTRSRPIINTRDEPHADSHSYRRLHVIVGDANMAEPSIALKVGSTLLVLEMIEADFGLPSLELANDIASIREISRDATGSTLLSLKDGTTMTALQIQQVVFEHASKWLEQRPEPEFSGTSNTEMARVLDLWGRMLKAIESGDFSEVDTEIDWVIKKKLIDRFIQRGNLGLDDPKLAQVDLTYHDIRPGRGLFSVLQSRGMIKRWTTDEAILAAVDTAPDTTRAHLRGRILKAADTLGVPVTVDWMRHKVNRPEPQSVELGDPFSAVNSEVDQLIEYMTVHAESYRS

SEQ ID NO: 25 - protein, 1950 aa, synthetic construct
  REGION 1..1950: note = Synthetic
MPKKKRKVGRVCRISSLRYRGPGIATMKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIAHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIANYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALEQRPQGGGGPKKKRKVGGSGMSTVESALTRRIMGIETEYGLTFVDGDSKKLRPDEIARRMFRPIVEKYSSSNIFIPNGSRLYLDVGSHPEYATAECDNLTQLINFEKAGDVIADRMAVDAEESLAKEDIAGQVYLFKNNVDSVGNSYGCHENYLVGRSMPLKALGKRLMPFLITRQLICGAGRIHHPNPLDKGESFPLGYCISQRSDHVWEGVSSATTRSRPIINTRDEPHADSHSYRRLHVIVGDANMAEPSIALKVGSTLLVLEMIEADFGLPSLELANDIASIREISRDATGSTLLSLKDGTTMTALQIQQVVFEHASKWLEQRPEPEFSGTSNTEMARVLDLWGRMLKAIESGDFSEVDTEIDWVIKKKLIDRFIQRGNLGLDDPKLAQVDLTYHDIRPGRGLFSVLQSRGMIKRWTTDEAILAAVDTAPDTTRAHLRGRILKAADTLGVPVTVDWMRHKVNRPEPQSVELGDPFSAVNSEVDQLIEYMTVHAESYRSEQKLISEEDLGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 26 - DNA, 21 bp, synthetic construct
  misc_feature 1..21: note = Synthetic
  source 1..21: mol_type = other DNA
actacaccgaggaaatgggct

SEQ ID NO: 27 - DNA, 22 bp, synthetic construct
  misc_feature 1..22: note = Synthetic
  source 1..22: mol_type = other DNA
cccacaatgccagttaagaaga

SEQ ID NO: 28 - DNA, 19 bp, synthetic construct
  misc_feature 1..19: note = Synthetic
  source 1..19: mol_type = other DNA
tgtccgtcagaacccatgc

SEQ ID NO: 29 - DNA, 21 bp, synthetic construct
  misc_feature 1..21: note = Synthetic
  source 1..21: mol_type = other DNA
aaagtcgaagttccatcgctc

SEQ ID NO: 30 - DNA, 20 bp, synthetic construct
  misc_feature 1..20: note = Synthetic
  source 1..20: mol_type = other DNA
cagaggaggtatgcagggag

SEQ ID NO: 31 - DNA, 20 bp, synthetic construct
  misc_feature 1..20: note = Synthetic
  source 1..20: mol_type = other DNA
ggatgtctagctccaagggg

SEQ ID NO: 32 - DNA, 21 bp, synthetic construct
  misc_feature 1..21: note = Synthetic
  source 1..21: mol_type = other DNA
catgtacgttgctatccaggc

SEQ ID NO: 33 - DNA, 21 bp, synthetic construct
  misc_feature 1..21: note = Synthetic
  source 1..21: mol_type = other DNA
ctccttaatgtcacgcacgat

SEQ ID NO: 34 - DNA, 22 bp, synthetic construct
  misc_feature 1..22: note = Synthetic
  source 1..22: mol_type = other DNA
agatccctccaaaatcaagtgg

SEQ ID NO: 35 - DNA, 21 bp, synthetic construct
  misc_feature 1..21: note = Synthetic
  source 1..21: mol_type = other DNA
ggcagagatgatgaccctttt

SEQ ID NO: 36 - DNA, 28 bp, synthetic construct
  misc_feature 1..28: note = Synthetic
  source 1..28: mol_type = other DNA
ctggcggcgggtgtggacgggcggcgga

SEQ ID NO: 37 - DNA, 28 bp, synthetic construct
  misc_feature 1..28: note = Synthetic
  source 1..28: mol_type = other DNA
tcggcaacctctttccatctagaagggc

SEQ ID NO: 38 - DNA, 28 bp, synthetic construct
  misc_feature 1..28: note = Synthetic
  source 1..28: mol_type = other DNA
atgataatgcaatagcaggacaggatga

SEQ ID NO: 39 - DNA, 28 bp, synthetic construct
  misc_feature 1..28: note = Synthetic
  source 1..28: mol_type = other DNA
tacactaagcacttcagtgcctccaggg

(19) United States
(12) Patent Application Publication
(10) Pub. No.: US 2022/0411771 A1
(43) Pub. Date: Dec. 29, 2022

(54) COMPOSITIONS AND METHODS FOR DETECTING NUCLEIC ACID-PROTEIN INTERACTIONS

(71) Applicant: ShanghaiTech University, Shanghai (CN)

(72) Inventors: Jilong Liu, Shanghai (CN); Ziheng Zhang, Shanghai (CN); Min Zhuang, Shanghai (CN); Weiping Sun, Shanghai (CN)

(21) Appl. No.: 17/822,418

(22) Filed: Aug. 25, 2022

Related U.S. Application Data
(63) Continuation of application No. PCT/CN2021/077602, filed on Feb. 24, 2021.

(30) Foreign Application Priority Data
Feb. 25, 2020 (CN) PCT/CN2020/076562

(51) Int. Cl. C12N 9/22; A01K 67/033; A01K 67/027; C12N 9/00; G01N 33/50

(52) U.S. Cl. CPC C12N 9/22; A01K 67/0339; A01K 67/0275; C12N 9/93; C12Y 603/01; G01N 33/5088; C07K 2319/00; C07K 2319/09; A01K 2217/05; A01K 2227/706; A01K 2227/105; A01K 2267/0393

(57) ABSTRACT

Compositions and methods for detecting nucleic acid-protein interactions, or more generally interactions between a nucleic acid and another molecule. A Cas protein (e.g., a catalytically dead Cas13) is fused to a proximity tagging enzyme (e.g., a Pup ligase) and thus brings the proximity tagging enzyme to the proximity of a protein that binds to a nucleic acid, when the Cas protein recognizes the nucleic acid, e.g., through a guide RNA. The proximity tagging enzyme then tags the protein enabling it to be identified as a protein that interacts with the nucleic acid.

### CROSS REFERENCE TO RELATED APPLICATIONS

This application is a continuation of International Patent Application No. PCT/CN2021/077602, filed Feb. 24, 2021, which claims the benefit of International Patent Application No. PCT/CN2020/076562, filed Feb. 25, 2020, the content of each of which is hereby incorporated by reference in its entirety.

### REFERENCE TO AN ELECTRONIC SEQUENCE LISTING

The contents of the electronic sequence listing (300694.xml; Size: 58,914 bytes; and Date of Creation: Aug. 24, 2022) is herein incorporated by reference in its entirety.

### BACKGROUND

Human cells encode a large number of RNAs, including many non-coding RNAs. These RNAs are expressed differentially in various cells and physiological conditions. However, the functions and regulatory mechanisms of the majority of these transcripts remain unknown. One potential key to understanding is the RNA-binding protein, which is a feature throughout the entire life cycle of RNA (including mRNA, lncRNA, etc.), indicating the importance of the study of detailed RNA-protein interactions.

RNA-binding proteins (RBPs) play important roles in various biological processes such as regulation, splicing, modification, localization, translation, and stabilization of RNAs. Many RNA-binding proteins, including some proteins that lack the classical RNA-binding domains, have distinct spatial and temporal distributions in cells and tissues. The malfunction of RBPs is responsible for many human diseases.

In order to gain insight into the function of RBPs, it is necessary to identify detailed interactions between an RNA and its binding proteins. Initially, the RNA immunoprecipitation (RIP) assay has been used to identify RNA-protein interactions, which was adapted from the chromatin immunoprecipitation assay (ChIP). However, because the RIP assay retains protein-protein interactions, it is not well suitable for studying direct RNA-protein contacts. To exploit zero-length covalent RNA-protein cross-linking and RNA fragmentation, a method named crosslinking and immunoprecipitation (CLIP) has been developed. By directly illuminating cells or tissues with UV-B light, it catalyzes the formation of covalent bonds between RNA and proteins that within the direct contact. Later, Photoactivatable-Ribonucleoside-Enhanced Crosslinking and Immunoprecipitation (PAR-CLIP) was developed to further improve the cross-linking efficiency of CLIP.

Another class of highly regarded methods named RNA antisense purification-mass spectrometry (RAP-MS) and comprehensive identification of RNA-binding proteins by mass spectrometry (ChIRP-MS) have been developed recently. Biotin-labeled DNA fragments complementary to the target RNA sequences were used to capture the target RNAs. RNA-protein complexes bind to the biotin-tagged DNA fragments, which were captured by streptavidin magnetic beads. The advantage of these mass spectrometry-based techniques is to capture RNA-protein interactions under natural conditions. However, it is difficult to design DNA fragments suitable for those experiments. Therefore, the desires for widely applicable detecting the RNA-protein interaction of specific RNAs for in vivo labeling without in vitro manipulation remain unfulfilled.

Moreover, it is also valuable to detect DNA-protein interactions as such interactions can impact the transcription and other activities of DNA fragments.

### SUMMARY

The present technology enables study of interactions between nucleic acids and nucleic acid-binding molecules. A Cas protein (e.g., a catalytically dead Cas13) is fused to a proximity tagging enzyme (e.g., a Pup ligase) and thus brings the proximity tagging enzyme to a nucleic acid, when the Cas protein recognizes the nucleic acid, e.g., with a guide RNA. The proximity tagging enzyme then tags the molecule enabling it to be identified as one that interacts with the nucleic acid.

In accordance with one embodiment of the present disclosure, therefore, provided is a non-human transgenic organism, comprising a recombinant polynucleotide in at least one cell of the organism, wherein the polynucleotide encodes a fusion protein comprising a clustered regularly interspaced short palindromic repeats (CRISPR)-associated (Cas) protein Cas13 and a proximity tagging enzyme.

In some embodiments, the polynucleotide further comprises an inducible promoter or a tissue-specific promoter that is operably linked to and regulates the expression of the fusion protein.

In another embodiment, provided is a method of identifying a protein that binds to a target RNA, comprising contacting activating the inducible promoter in the non-human transgenic organism in the presence of a guide RNA that is specific to the target RNA, under conditions to allow the Cas13 protein to bind to the target RNA and the proximity tagging enzyme to tag proteins bound to the target RNA.

Also provided a fusion protein comprising a clustered regularly interspaced short palindromic repeats (CRISPR)-associated (Cas) protein Cas13 and a proximity tagging enzyme. In some embodiments, the Cas13 is selected from the group consisting of Cas13a, Cas13b, Cas13c, and Cas13d. Examples include LshCas13a, LwaCas13a, LseCas13a, LbmCas13a, LbnCas13a, CamCas13a, CgaCas13a, Cga2Cas13a, Pprcas13a, LweCas13a, LbfCas13a, Lwa2cas13a, RcsCas13a, RcrCas13a, RcdCas13a, LbuCas13a, HheCas13a, EreCas13a, EbaCas13a, BmaCas13a, LspCas13a, BzoCas13b, PinCas13b, PbuCas13b, AspCas13b, PsmCas13b, RanCas13b, PauCas13b, PsaCas13b, Pin2Cas13b, CcaCas13b, PguCas13b, PspCas13b, FbrCas13b, PgiCas13b, Pin3Cas13b, FnsCas13c, FndCas13c, FnbCas13c, FnfCas13c, FpeCas13c, FulCas13c, AspCas13c, UrCas13d, RffCas13d, RaCas13d, AdmCas13d, PIE0Cas13d, EsCas13d, and RfxCas13d. In some embodiments, the Cas13 is catalytically dead, such as dLwCas13a with an R474A or R1046A mutation.

In some embodiments, the proximity tagging enzyme is selected from the group consisting of a Pup ligase, a biotin ligase, and an ascorbate peroxidase. In some embodiments, the proximity tagging enzyme is PafA, TurboID, or MiniTurbo.

### BRIEF DESCRIPTION OF THE DRAWINGS

FIG. 1 illustrates an example design of CRUIS. A, Schematic of the CRISPR-based RNA targeting, proximity targeting system. PafA is fused to dLwaCas13a protein and mediates PupE modification of the surrounding proteins of the target RNA. B, Plasmids involved in CRUIS. C, Timeline for CRUIS to capture RNA-protein interaction.

FIG. 2 presents the results of the testing the activity of CRUIS. A, HEK239T cells were co-transfected with LwaCas13a-PafA and sgRNA expression plasmid to detect the mRNA expression level of the target gene after 24 hours; non-target sgRNA was used as the negative control (n=3, mean±S.E.M). B, Plasmids used in this assay. C, Representative immunofluorescence images of 293T-CRUIS cells treated with 100 mM sodium malonate (scale bar 10 μm). Stress granules are indicated by G3BP1 staining. D, Testing the proximity label activity of CRUIS.

FIG. 3 shows capturing RNA-binding proteins of NORAD by CRUIS. A, The target RBPs were determined by a moderated t-test (p value<0.05) and fold change (fold change>3). B, Bar plot of log 2 fold change (log 2FC) of the identified proteins in NORAD interactome by CRIUS. C, The top 15 GO-enriched biological processes of proteins in NORAD interactome by CRUIS (red dots), the negative control (green dots) and combined datasets (light blue dots). (p. value<0.01, p. adjust<0.05) D, Subcellular distribution of the identified proteins in NORAD interactome by CRIUS. E, Comparison of NORAD interactome by CRUIS with the two public datasets: RAP MS and StarBase v2.0 database.

FIG. 4 shows validation of proteins enriched by RIP-qPCR. A. The pattern diagram shows that the marker protein is HA-tag at the C-terminus for subsequent RIP. B. Schematic of RNA immunoprecipitation for quantification of RNA-protein interaction. C. Some proteins found by CRUIS could significantly enrich NORAD transcript compared with the anti-IgG group and control (n=3, mean±S.E.M. ***P<0.001; **P<0.01; *P<0.05).

FIG. 5 illustrates a workflow of CRUIS to identify the RNA-protein interactions. Cells were cultured in 150 mm dishes; 12 hours after transfection (sgRNA and pCMV-Bio-PupE) biotin was added to make the final conc. 20 μM; 24 hours after addition of biotin the cells were collected and lysed. Streptavidin-beads were used for enriching and purifying proteins labeled with Bio-PupE. Finally, the type and abundance of proteins were identified by protein mass spectrometry after digestion by trypsin.

FIG. 6 shows a diagram of the CRUIS plasmid. NLS, nuclear localization sequence; pCAG, CAG promoter; myc, myc epitope tag; P2A, P2A self-cleaving peptide; EGFP, enhanced green fluorescent protein; ITRs, inverted terminal repeats. Thus, the fusion gene is currently too large for viral transduction. We obtained cell lines with stable expression of CRUIS using the piggyBac transposon system. Although the transfection efficiency was low, the GFP-positive cells were enriched by sorting. Single colonies were picked, expanded and tested.

FIG. 7 shows subcellular localization of CRUIS. (A) Schematic diagram of the plasmid structure used in this assay, EGFP was used to label CRUIS in the C-terminus (no P2A between CRUIS and EGFP in the construct). (B) After transfected pCAG-CRUIS-EGFP for 24 h, the location of CRUIS was determined by EGFP. The results showed distribution in the nucleus and cytoplasm (scale bar 10 μm).

FIG. 8 illustrates selection of CRUIS stable cell lines. (A) Anti-myc western blotting shows 10 clones with stable expression of CRUIS. (B) Three CRUIS stable cell lines, P2, P7, and P8, were selected to test the enzyme activity of PafA in CRUIS. Anti-streptavidin western blotting indicates that CRUIS shows reliable proximity targeting activity.

FIG. 9 shows expression levels of RNAs. HEK239T cells are co-transfected with LwaCas13a-PafA and sgRNA expression plasmid to detect the mRNA expression level of the target gene after 24 hours. The resulting values were normalized to GAPDH expressions. (n=3, mean±S.E.M ***P<0.001; **P<0.01; *P<0.05).

FIG. 10 shows obtaining the RNA-binding proteins of P21 mRNA by CRUIS. (A) RNA-binding proteins of P21 mRNA were captured by CRUIS. Some proteins were enriched in the P21 group (p21-target sgRNA) compared with control (non-target sgRNA). Some of these were p21-binding proteins identified previously (marked in red). The red dots in the scatterplot are examples of known P21 RNA-binding proteins in StarBase v2.0 database. (B) Western blot showed CRUIS-mediated Bio-PupE modification of HNRNPK. After capturing the RBPs of p21 mRNA by CRUIS, the labeled proteins were enriched using streptavidin magnetic beads, and HNRNPK was detected by HNRNPK-specific antibody. Compared to the non-target sgRNA group, the p21-target group showed highly enriched of HNRNPK.

FIG. 11A-D illustrate processes for preparing transgenic organisms (mice and fruit flies) useful for detecting RNA-binding proteins with the CRUIS technology.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

The term “isolated” as used herein with respect to cells, nucleic acids, such as DNA or RNA, refers to molecules separated from other DNAs or RNAs, respectively, that are present in the natural source of the macromolecule. The term “isolated” as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to cells or polypeptides which are isolated from other cellular proteins or tissues. Isolated polypeptides is meant to encompass both purified and recombinant polypeptides.

As used herein, the term “recombinant” as it pertains to polypeptides or polynucleotides intends a form of the polypeptide or polynucleotide that does not exist naturally, a non-limiting example of which can be created by combining polynucleotides or polypeptides that would not normally occur together.

### Detection of Nucleic Acid-Protein Interactions

The experimental example has tested a system for detecting RNA-protein interactions, which is referred to as CRISPR-based RNA-United Interacting System (CRUIS), which uses the CRISPR-based RNA-target Cas nuclease as an RNA tracker to bring the proximity-labeling system to a designated target RNA. CRUIS can capture RNA-protein interactions of specific RNA sequences effectively. In CRUIS, a dead RNA-guided RNA targeting nuclease, e.g., LwaCas13a (dLwaCas13a), is used as a tracker to target specific RNA sequences, while a proximity enzyme, e.g., PafA, is fused to the nuclease to label any surrounding RNA-binding proteins. The labeled proteins can then be enriched and identified.

Using this technology, proteins that interact with specific RNAs can be labeled in living cells, which avoids the risk of RNA degradation introduced by processing RNA-protein complexes in vitro. In addition, this technology can avoid over-expressing the target RNA with the MS2-tag sequence in the cell, so the abundance of the target RNA in the cell is in a natural state and the acquired RNA is closer to the real situation.

In comparison to the conventional methods, CRUIS shows quite a few advantages. First, it provides a simple and effective way to obtain potential RNA-binding proteins of target RNA. Second, CRUIS can identify RNA-protein interactions in a natural state. Finally, CRUIS can label potential RNA-binding proteins in living cells, thereby avoiding the manipulation of RNA in vitro and decreasing the impact of RNA degradation. CRUIS can be universally used for different types of RNA, including lncRNA and mRNA, indicating that CRUIS has broad applicability. Furthermore, when using a DNA-targeting Cas protein, such as Cas9 and Cas12a/b, the technology can be useful for detecting DNA-protein interactions.

### Fusion Proteins Useful for Detecting Nucleic Acid-Molecule Interactions

One embodiment of the present disclosure provides compositions and methods for detecting nucleic acid-molecule interactions. The present technology, in some embodiments, employs a fusion protein that includes a Cas protein and a proximity tagging enzyme. The Cas protein, through the use of an appropriate guide RNA, can selectively bind a nucleic acid molecule. Once bound, the proximity tagging enzyme can, under suitable conditions and with suitable substrates, tag molecules that interact with the nucleic acid and thus identifying those molecules with mass spectrometry.

In one embodiment, the present disclosure provides a fusion protein comprising a clustered regularly interspaced short palindromic repeats (CRISPR)-associated (Cas) protein and a proximity tagging enzyme.

The term “Cas protein” or “clustered regularly interspaced short palindromic repeats (CRISPR)-associated (Cas) protein” refers to RNA-guided DNA/RNA endonuclease enzymes associated with the CRISPR (Clustered Regularly Interspaced Short Palindromic Repeats) adaptive immunity system in Streptococcus pyogenes, as well as other bacteria. Cas proteins include Cas9 proteins, Cas12a (Cpf1) proteins, Cas12b (formerly known as C2c1) proteins, Cas13 proteins and various engineered counterparts.

Example DNA-targeting Cas proteins include SpCas9, FnCas9, St1Cas9, St3Cas9, NmCas9, SaCas9, AsCpf1, LbCpf1, FnCpf1, VQR SpCas9, EQR SpCas9, VRER SpCas9, SpCas9-NG, xSpCas9, RHA FnCas9, KKH SaCas9, NmeCas9, StCas9, CjCas9, AsCpf1, FnCpf1, SsCpf1, PcCpf1, BpCpf1, CmtCpf1, LiCpf1, PmCpf1, Pb3310Cpf1, Pb4417Cpf1, BsCpf1, EeCpf1, BhCas12b, AkCas12b, EbCas12b, LsCas12b and those provided in Table A below.

TABLE A
Example DNA-Targeting Cas Proteins

| Cas | |
|---|---|
| protein | |
| types | Cas proteins |
| Cas9 | Cas9 from Staphylococcus aureus (SaCas9) |
| proteins | Cas9 from Neisseria meningitidis (NmeCas9) |
| | Cas9 from Streptococcus thermophilus (StCas9) |
| | Cas9 from Campylobacter jejuni (CjCas9) |
| Cas12a | Cas12a (Cpf1) from Acidaminococcus sp BV3L6 (AsCpf1) |
| (Cpf1) | Cas12a (Cpf1) from Francisella novicida sp BV3L6 (FnCpf1) |
| proteins | Cas12a (Cpf1) from Smithella sp SC K08D17 (SsCpf1) |
| | Cas12a (Cpf1) from Porphyromonas crevioricanis (PcCpf1) |
| | Cas12a (Cpf1) from Butyrivibrio proteoclasticus (BpCpf1) |
| | Cas12a (Cpf1) from Candidatus Methanoplasma termitum |
| | (CmtCpf1) |
| | Cas12a (Cpf1) from Leptospira inadai (LiCpf1) |
| | Cas12a (Cpf1) from Porphyromonas macacae (PmCpf1) |
| | Cas12a (Cpf1) from Peregrinibacteria bacterium GW2011 |
| | WA2 33 10 (Pb3310Cpf1) |
| | Cas12a (Cpf1) from Parcubacteria bacterium GW2011 GWC2 |
| | 44 17 (Pb4417Cpf1) |
| | Cas12a (Cpf1) from Butyrivibrio sp. NC3005 (BsCpf1) |
| | Cas12a (Cpf1) from Eubacterium eligens (EeCpf1) |
| Cas12b | Cas12b (C2c1) Bacillus hisashii (BhCas12b) |
| (C2c1) | Cas12b (C2c1) Bacillus hisashii with a gain-of-function |
| proteins | mutation |
| | (see, e.g., Strecker et al., Nature Communications 10 |
| | (article 212) (2019) |
| | Cas12b (C2c1) Alicyclobacillus kakegawensis (AkCas12b) |
| | Cas12b (C2c1) Elusimicrobia bacterium (EbCas12b) |
| | Cas12b (C2c1) Laceyella sediminis (Ls) (LsCas12b) |

In some embodiments, the Cas protein is a DNA-targeting Cas protein, such as Cas9, Cas12a and Cas12b. In some embodiments, the Cas protein is a RNA-targeting Cas protein, such as Cas13.

Cas13 targets RNA. The Cas13 family contains at least four known subtypes, including Cas13a (formerly C2c2), Cas13b, Cas13c, and Cas13d, classified based on the identity of the Cas13 protein and additional locus features. All known Cas13 family members contain two HEPN domains, which confer RNase activity. Cas13 can be reprogrammed to cleave a targeted ssRNA molecule through a short guide RNA with complementarity to the target sequence.

Cas13s function similarly to Cas9, using a ˜64-nt guide RNA to encode target specificity. The Cas13 protein complexes with the guide RNA via recognition of a short hairpin in the crRNA, and target specificity is encoded by a 28-30-nt spacer that is complementary to the target region. In addition to programmable RNase activity, Cas13s can also exhibit collateral activity after recognition and cleavage of a target transcript, leading to non-specific degradation of any nearby transcripts regardless of complementarity to the spacer.

Non-limiting examples of Cas13 proteins are listed in the table below.

TABLE B
Example RNA-Targeting Cas Proteins

| Subtype | Name | Host Organism | Protein Accession or Sequence |
|---|---|---|---|
| Cas13a | LshCas13a | Leptotrichia shahii | WP_018451595.1 |
| | LwaCas13a | Leptotrichia wadei | WP_021746774.1 |
| | LseCas13a | Listeria seeligeri | WP_012985477.1 |
| | LbmCas13a | Lachnospiraceae bacterium MA2020 | WP_044921188.1 |
| | LbnCas13a | Lachnospiraceae bacterium NK4A179 | WP_022785443.1 |
| | CamCas13a | [Clostridium] aminophilum DSM 10710 | WP_031473346.1 |
| | CgaCas13a | Carnobacterium gallinarum DSM 4847 | WP_034560163.1 |
| | Cga2Cas13a | Carnobacterium gallinarum DSM 4847 | WP_034563842.1 |
| | PprCas13a | Paludibacter propionicigenes WB4 | WP_013443710.1 |
| | LweCas13a | Listeria weihenstephanensis FSL R9-0317 | WP_036059185.1 |
| | LbfCas13a | Listeriaceae bacterium FSL M6-635 | WP_036091002.1 |
| | Lwa2Cas13a | Leptotrichia wadei F0279 | WP_021746774.1 |
| | RcsCas13a | Rhodobacter capsulatus SB 1003 | WP_013067728.1 |
| | RcrCas13a | Rhodobacter capsulatus R121 | WP_023911507.1 |
| | RcdCas13a | Rhodobacter capsulatus DE442 | WP_023911507.1 |
| | LbuCas13a | Leptotrichia buccalis C-1013-b | WP_015770004.1 |
| | HheCas13a | Herbinix hemicellulosilytica | CRZ35554.1 |
| | EreCas13a | [Eubacterium] rectale | WP_055061018.1 |
| | EbaCas13a | Eubacteriaceae bacterium CHKCI004 | WP_090127496.1 |
| | BmaCas13a | Blautia sp. Marseille-P2398 | WP_062808098.1 |
| | LspCas13a | Leptotrichia sp. oral taxon 879 str.F0557 | WP_021744063.1 |
| Cas13b | BzoCas13b | Bergeyella zoohelcum | WP_002664492 |
| | PinCas13b | Prevotella intermedia | WP_036860899 |
| | PbuCas13b | Prevotella buccae | WP_004343973 |
| | AspCas13b | Alistipes sp. ZOR0009 | WP_047447901 |
| | PsmCas13b | Prevotella sp. MA2016 | WP_036929175 |
| | RanCas13b | Riemerella anatipestifer | WP_004919755 |
| | PauCas13b | Prevotella aurantiaca | WP_025000926 |
| | PsaCas13b | Prevotella saccharolytica | WP_051522484 |
| | Pin2Cas13b | Prevotella intermedia | WP_061868553 |
| | CcaCas13b | Capnocytophaga canimorsus | WP_013997271 |
| | PguCas13b | Porphyromonas gulae | WP_039434803 |
| | PspCas13b | Prevotella sp. P5-125 | WP_044065294 |
| | FbrCas13b | Flavobacterium branchiophilum | WP_014084666 |
| | PgiCas13b | Porphyromonas gingivalis | WP_053444417 |
| | Pin3Cas13b | Prevotella intermedia | WP_050955369 |
| Cas13c | FnsCas13c | Fusobacterium necrophorum subsp. | WP_005959231.1 |
| | | funduliforme ATCC 51357 | |
| | | contig00003 | |
| | FndCas13c | Fusobacterium necrophorum DJ-2 | WP_035906563.1 |
| | | contig0065, whole genome shotgun | |
| | | sequence | |
| | FnbCas13c | Fusobacterium necrophorum BFTR-1 | WP_035935671.1 |
| | | contig0068 | |
| | FnfCas13c | Fusobacterium necrophorum subsp. | EHO19081.1 |
| | | funduliforme 1_1_36S contl.14 | |
| | FpeCas13c | Fusobacterium perfoetens ATCC | WP_027128616.1 |
| | | 29250 T364DRAFT_scaffold00009.9_C | |
| | FulCas13c | Fusobacterium ulcerans ATCC | WP_040490876.1 |
| | | 49185 cont2.38 | |
| | AspCas13c | Anaerosalibacter sp. ND1 genome | WP_042678931.1 |
| | | assembly Anaerosalibacter | |
| | | massiliensis ND1 | |
| Cas13d | UrCas13d | Uncultured Ruminoccocus sp. | MAKKNKMKPRELREAQKKARQLKAAEINNNAAPAIA |
| | | | AMPAAEVIAPAAEKKKSSVKAAGMKSILVSENKMYI |
| | | | TSFGKGNSAVLEYEVDNNDYNQTQLSSKDNSNIQLG |
| | | | GVNEVNITFSSKHGFESGVEINTSNPTHRSGESSPV |
| | | | RGDMLGLKSELEKRFFGKTFDDNIHIQLIYNILDIE |
| | | | KILAVYVTNIVYALNNMLGVKGSESHDDFIGYLSTN |
| | | | NIYDVFIDPDNSSLSDDKKANVRKSLSKFNALLKTK |
| | | | RLGYFGLEEPKTKDNRVSQAYKKRVYHMLAIVGQIR |
| | | | QCVFHDKSGAKRFDLYSFINNIDPEYRDTLDYLVEE |
| | | | RLKSINKDFIEDNKVNISLLIDMMKGYEADDIIRLY |
| | | | YDFIVLKSQKNLGFSIKKLREKMLDEYGFRFKDKQY |
| | | | DSVRSKMYKLMDFLLFCNYYRNDIAAGESLVRKLRF |
| | | | SMTDDEKEGIYADEAAKLWGKFRNDFENIADHMNGD |
| | | | VIKELGKADMDFDEKILDSEKKNASDLLYFSKMIYM |
| | | | LTYFLDGKEINDLLTTLISKFDNIKEFLKIMKSSAV |
| | | | DVECELTAGYKLFNDSQRITNELFIVKNIASMRKPA |
| | | | ASAKLTMFRDALTILGIDDKITDDRISGILKLKEKG |
| | | | KGIHGLRNFITNNVIESSRFVYLIKYANAQKIREVA |
| | | | KNEKVVMFVLGGIPDTQIERYYKSCVEFPDMNSSLG |
| | | | VKRSELARMIKNISFDDFKNVKQQAKGRENVAKERA |
| | | | KAVIGLYLTVMYLLVKNLVNVNARYVIAIHCLERDF |
| | | | GLYKEIIPELASKNLKNDYRILSQTLCELCDKSPNL |
| | | | FLKKNERLRKCVEVDINNADSSMTRKYRNCIAHLTV |
| | | | VRELKEYIGDICTVDSYFSIYHYVMQRCITKRENDT |
| | | | KQEEKIKYEDDLLKNHGYTKDFVKALNSPFGYNIPR |
| | | | FKNLSIEQLFDRNEYLTEK |
| | | | (SEQ ID NO: 13) |
| | RffCas13d | Ruminoccocus flavefaciens FDI | MKKKMSLREKREAEKQAKKAAYSAASKNTDSKPAEK |
| | | | KAETPKPAEIISDNSRNKTAVKAAGLKSTIISGDKL |
| | | | YMTSFGKGNAAVIEQKIDINDYSFSAMKDTPSLEVD |
| | | | KAESKEISFSSHHPFVKNDKLTTYNPLYGGKDNPEK |
| | | | PVGRDMLGLKDKLEERYFGCTFNDNLHIQIIYNILD |
| | | | IEKILAVHSANITTALDHMVDEDDEKYLNSDYIGYM |
| | | | NTINTYDVFMDPSKNSSLSPKDRKNIDNSRAKFEKL |
| | | | LSTKRLGYFGFDYDANGKDKKKNEEIKKRLYHLTAF |
| | | | AGQLRQWSFHSAGNYPRTWLYKLDSLDKEYLDTLDH |
| | | | YFDKRFNDINDDFVTKNATNLYILKEVFPEANFKDI |
| | | | ADLYYDFIVIKSHKNMGFSIKKLREKMLECDGADRI |
| | | | KEQDMDSVRSKLYKLIDFCIFKYYHEFPELSEKNVD |
| | | | ILRAAVSDTKKDNLYSDEAARLWSIFKEKFLGFCDK |
| | | | IVVWVTGEHEKDITSVIDKDAYRNRSNVSYFSKLMY |
| | | | AMCFFLDGKEINDLLTTLINKFDNIANQIKTAKELG |
| | | | INTAFVKNYDFFNHSEKYVDELNIVKNIARMKKPSS |
| | | | NAKKAMYHDALTILGIPEDMDEKALDEELDLILEKK |
| | | | TDPVTGKPLKGKNPLRNFIANNVIENSRFIYLIKFC |
| | | | NPENVRKIVNNTKVTEFVLKRIPDAQIERYYKSCTD |
| | | | SEMNPPTEKKITELAGKLKDMNFGNFRNVRQSAKEN |
| | | | MEKERFKAVIGLYLTVVYRVVKNLVDVNSRYIMAFH |
| | | | SLERDSQLYNVSVDNDYLALTDTLVKEGDNSRSRYL |
| | | | AGNKRLRDCVKQDIDNAKKWFVSDKYNSITKYRNNV |
| | | | AHLTAVRNCAEFIGDITKIDSYFALYHYLIQRQLAK |
| | | | GLDHERSGFDRNYPQYAPLFKWHTYVKDVVKALNAP |
| | | | FGYNIPRFKNLSIDALFDRNEIKKNDGEKKSDD |
| | | | (SEQ ID NO: 14) |
| | RaCas13d | Ruminoccocus albus | MAKKSKGMSLREKRELEKQKRIQKAAVNSVNDTPEK |
| | | | TEEANVVSVNVRTSAENKHSKKSAAKALGLKSGLVI |
| | | | GDELYLTSFGRGNEAKLEKKISGDTVEKLGIGAFEV |
| | | | AERDESTLTLESGRIKDKTARPKDPRHITVDTQGKF |
| | | | KEDMLGIRSVLEKKIFGKTFDDNIHVQLAYNILDVE |
| | | | KIMAQYVSDIVYMLHNTDKTERNDNLMGYMSIRNTY |
| | | | KTFCDTSNLPDDTKQKVENQKREFDKIIKSGRLGYF |
| | | | GEAFMVNSGNSTKLRPEKEIYHIFALMASLRQSYFH |
| | | | GYVKDTDYQGTTWAYTLEDKLKGPSHEFRETIDKIF |
| | | | DEGFSKISKDFGKMNKVNLQILEQMIGELYGSIERQ |
| | | | NLTCDYYDFIQLKKHKYLGFSIKRLRETMLETTPAE |
| | | | CYKAECYNSERQKLYKLIDFLIYDLYYNRKPARISE |
| | | | IVDKLRESVNDEEKESIYSVEAKYVYESLSKVLDKS |
| | | | LKNSVSGETIKDLQKRYDDETANRIWDISQHSISGN |
| | | | VNCFCKLIYIMTLMLDGKEINDLLTTLVNKFDNIAS |
| | | | FIDVMDELGLEHSFTDNYKMFADSKAICLDLQFINS |
| | | | FARMSKIDDEKSKRQLFRDALVILDIGNKDETWINN |
| | | | YLDSDIFKLDKEGNKLKGARHDFRNFIANNVIKSSR |
| | | | FKYLVKYSSADGMIKLKTNEKLIGFVLDKLPETQID |
| | | | RYYESCGLDNAVVDKKVRIEKLSGLIRDMKFDDFSG |
| | | | VKTSNKAGDNDKQDKAKYQAIISLYLMVLYQIVKNM |
| | | | IYVNSRYVIAFHCLERDFGMYGKDFGKYYQGCRKLT |
| | | | DHFIEEKYMKEGKLGCNKKVGRYLKNNISCCTDGLI |
| | | | NTYRNQVDHFAVVRKIGNYAAYIKSIGSWFELYHYV |
| | | | IQRIVFDEYRFALNNTESNYKNSIIKHHTYCKDMVK |
| | | | ALNTPFGYDLPRYKNLSIGDLFDRNNYLNKTKESID |
| | | | ANSSIDSQ |
| | | | (SEQ ID NO: 15) |
| | AdmCas13d | Anaerobic digester metagenome 15706 | MNNKRKTKAKAAGLKSVFFDQKQAVLTTFAKGNNSQ |
| | | | IEKKVVNSEVKDLRQPPAFDLELKEKTFYISGKNNI |
| | | | NTSRENPLASASLPLSKRQRIRAERIKRAREENRPY |
| | | | HNVKRVGEDDLRAKADLEKHYFGKEYSDNLKIQIIY |
| | | | NILDINKIISPYINDIVYSMNNLARNDEYIDGKIDV |
| | | | IGSLSSTTDYSSFMSPNKDLEKEKKFSFHRENYKKF |
| | | | VEASKPYMRYYGKVFIRDVKKSKLSTGKGEKIEVMY |
| | | | RSDEEIFTIFQILSYVRQSIMHNDIGNKSSILAIEK |
| | | | YPARFVGFLSDLLKTKTNDVNRMFIDNNSQTNFWVL |
| | | | FSIFGLQDHTSGADKICRNFYDFVIKADSKNLGFSL |
| | | | KKIRELMLDLPNANMLRDHQFDTVRSKFYTLLDFI1 |
| | | | YQHYLEEKSRIDNMVEKLRMTLKEEEKEVLYAAEAK |
| | | | IVWNAIGAKVINKLVPMMNGDALKE1KRKNRDRKLP |
| | | | QSVIATVQVNSDANVFSGLIYFLTLFLDGKEINEMV |
| | | | SNLITKFENIDSLLHVDREIYKSDEKDLDLEIEKLA |
| | | | LFFKGVVRPNAKTDTGAGEISKSFSIFQSAERIIEE |
| | | | LKFIKNVTRMDNEIFPSEGVFLDAANVLGVRGDDFD |
| | | | FSNEFVGDDLHSDANKKIINKINGTKEDRNLRNFI1 |
| | | | NNVVKSRRFQYIARHMNTHYVKQLANNETLNRFVLN |
| | | | KMGDAKIINRYYESISGNTPNIEVRSQIDYLVKRLR |
| | | | SFSFEDLNDVKQKVRPGTNESIEKEKKKALVGLCLT |
| | | | IQYLVYKNLVNINARYTTAFYCLERDSKLKGFGVDV |
| | | | WRDFESYTALTNHFIKEGYLPVRKAEILRANLKHLD |
| | | | CEDGFKYYRNQVTHLNAIRVAYKYINEIKSVHSYFA |
| | | | LYHYIMQRHLYDSLQAKAKDSSGFVIDALKKSFEHK |
| | | | IYSKDLLHVLHSPFGYNTARYKNLSIEALFDKNESR |
| | | | PEVNPLSTND |
| | | | (SEQ ID NO: 16) |
| | PIE0Cas13d | Gut metagenome assembly P1E0-k21 | MEREVKKPPKKSLAKAAGLKSTFVISPQEKELAMTA |
| | | | FGRGNDALLQKRIVDGVVRDVAGEKQQFQVQRQDES |
| | | | RFRLQNSRLADRTVTADDPLHRAETPRRQPLGAGMD |
| | | | QLRRKAILEQKYFGRTFDDNIHIQLIYNILDIHKML |
| | | | AVPANHIVHTLNLLGGYGETDFVGMLPAGLPYDKLR |
| | | | VVKKKNGDTVDIKADIAAYAKRPQLAYLGAAFYDVT |
| | | | PGKSKRDAARGRVKREQDVYAILSLMSLLRQFCARD |
| | | | SVRIWGQNTTAALYHLQALPQDMKDLLDDGWRRALG |
| | | | GVNDHFLDTNKVNLLTLFEYYGAETKQARVALTQDF |
| | | | YRFVVLKEQKNMGFSLRRLREELLKLPDAAYLTGQE |
| | | | YDSVRQKLYMLLDFLLCRLYAQERADRCEELVSALR |
| | | | CALSDEEKDTVYQAEAAALWQALGDTLRRKLLPLLK |
| | | | GKKLQDKDKKKSDELGLSRDVLDGVLFRPAQQGSRA |
| | | | NADYFCRLMHLSTWFMDGKEINTLLTTLISKLENID |
| | | | SLRSVLESMGLAYSFVPAYAMFDHSRYIAGQLRVVN |
| | | | NIARMRKPAIGAKREMYRAAVVLLGVDSPEAAAAIT |
| | | | DDLLQIDPETGKVRPRSDSARDTGLRNFIANNVVES |
| | | | RRFTYLLRYMTPEQARVLAQNEKLIAFVLSTVPDTQ |
| | | | LERYCRTCGREDITGRPAQIRYLTAQIMGVRYESFT |
| | | | DVEQRGRGDNPKKERYKALIGLYLTVLYLAVKNMVN |
| | | | CNARYVIAFYCRDRDTALYQKEVCWYDLEEDKKSGK |
| | | | QRQVEDYTALTRYFVSQGYLNRHACGYLRSNMNGIS |
| | | | NSLLTAYRNAVDHLNAIPPLGSLCRDIGRVDSYFAL |
| | | | YHYAVQQYLNGRYYRKTPREQELFAAMAQHRTWCSD |
| | | | LVKALNTPFGYNLARYKNLSIDGLFDREGDHVVRED |
| | | | GEKPAE |
| | | | (SEQ ID NO: 17) |
| | EsCas13d | Eubacterium siraeum DSM15702 | MGKKIHARDLREQRKTDRTEKFADQNKKREAERAVP |
| | | | KKDAAVSVKSVSSVSSKKDNVTKSMAKAAGVKSVFA |
| | | | VGNTVYMTSFGRGNDAVLEQKIVDTSHEPLNIDDPA |
| | | | YQLNVVTMNGYSVTGHRGETVSAVTDNPLRRFNGRK |
| | | | KDEPEQSVPTDMLCLKPTLEKKFFGKEFDDNIHIQL |
| | | | IYNILDIEKILAVYSTNAIYALNNMSADENIENSDF |
| | | | FMKRTTDETFDDFEKKKESTNSREKADFDAFEKFIG |
| | | | NYRLAYFADAFYVNKKNPKGKAKNVLREDKELYSVL |
| | | | TLIGKLRHWCVHSEEGRAEFWLYKLDELKDDFKNVL |
| | | | DVVYNRPVEEINNRFIENNKVNIQILGSVYKNTDIA |
| | | | ELVRSYYEFLITKKYKNMGFSIKKLRESMLEGKGYA |
| | | | DKEYDSVRNKLYQMTDFILYTGYINEDSDRADDLVN |
| | | | TLRSSLKEDDKTTVYCKEADYLWKKYRESIREVADA |
| | | | LDGDNIKKLSKSNIEIQEDKLRKCFISYADSVSEFT |
| | | | KLIYLLTRFLSGKEINDLVTTLINKFDNIRSFLEIM |
| | | | DELGLDRTFTAEYSFFEGSTKYLAELVELNSFVKSC |
| | | | SFDINAKRTMYRDALDILGIESDKTEEDIEKMIDNI |
| | | | LQIDANGDKKLKKNNGLRNFIASNVIDSNRFKYLVR |
| | | | YGNPKKIRETAKCKPAVRFVLNEIPDAQIERYYEAC |
| | | | CPKNTALCSANKRREKLADMIAEIKFENFSDAGNYQ |
| | | | KANVTSRTSEAE1KRKNQAIIRLYLTVMYIMLKNLV |
| | | | NVNARYVIAFHCVERDTKLYAESGLEVGNIEKNKTN |
| | | | LTMAVMGVKLENGIIKTEFDKSFAENAANRYLRNAR |
| | | | WYKLILDNLKKSERAVVNEFRNTVCHLNAIRNININ |
| | | | IKEIKEVENYFALYHYLIQKHLENRFADKKVERDTG |
| | | | DFISKLEEHKTYCKDFVKAYCTPFGYNLVRYKNLTI |
| | | | DGLFDKNYPGKDDSDEQK |
| | | | (SEQ ID NO: 18) |
| | RfxCas13d | Ruminoccocus flavefaciens XPD3002 | MIEKKKSFAKGMGVKSTLVSGSKVYMTTFAEGSDAR |
| | | | LEKIVEGDSIRSVNEGEAFSAEMADKNAGYKIGNAK |
| | | | FSHPKGYAVVANNPLYTGPVQQDMLGLKETLEKRYF |
| | | | GESADGNDNICIQVIHNILDIEKILAEYITNAAYAV |
| | | | NNISGLDKDIIGFGKFSTVYTYDEFKDPEHHRAAFN |
| | | | NNDKLINAIKAQYDEFDNFLDNPRLGYFGQAFFSKE |
| | | | GRNY11NYGNECYDILALLSGLRHWVVHNNEEESRI |
| | | | SRTWLYNLDKNLDNEYISTLNYLYDRITNELTNSFS |
| | | | KNSAANVNYIAETLGINPAEFAEQYFRFSIMKEQKN |
| | | | LGFNITKLREVMLDRKDMSEIRKNHKVFDSIRTKVY |
| | | | TMMDFVIYRYYIEEDAKVAAANKSLPDNEKSLSEKD |
| | | | IFVINLRGSFNDDQKDALYYDEANRIWRKLENIMHN |
| | | | IKEFRGNKTREYKKKDAPRLPRILPAGRDVSAFSKL |
| | | | MYALTMFLDGKEINDLLTTLINKFDNIQSFLKVMPL |
| | | | IGVNAKFVEEYAFFKDSAKIADELRLIKSFARMGEP |
| | | | IADARRAMYIDAIRILGTNLSYDELKALADTFSLDE |
| | | | NGNKLKKGKHGMRNF11NNVISNKRFHYLIRYGDPA |
| | | | HLHEIAKNEAVVKFVLGRIADIQKKQGQNGKNQIDR |
| | | | YYETCIGKDKGKSVSEKVDALTKIITGMNYDQFDKK |
| | | | RSVIEDTGRENAEREKFKKIISLYLTVIYHILKNIV |
| | | | NINARYVIGFHCVERDAQLYKEKGYDINLKKLEEKG |
| | | | FSSVTKLCAGIDETAPDKRKDVEKEMAERAKESIDS |
| | | | LESANPKLYANYIKYSDEKKAEEFTRQINREKAKTA |
| | | | LNAYLRNTKWNVIIREDLLRIDNKTCTLFRNKAVHL |
| | | | EVARYVHAYINDIAEVNSYFQLYHYIMQRIIMNERY |
| | | | EKSSGKVSEYFDAVNDEKKYNDRLLKLLCVPFGYCI |
| | | | PRFKNLSIEALFDRNEAAKFDKEKKKVSGNS |
| | | | (SEQ ID NO: 19) |

The Cas protein, in some embodiments, is catalytically inactive/dead. Catalytically dead Cas proteins can be readily prepared by mutating one or more amino acid residues in the Cas protein's catalytic domain. Dead Cas9, Cas12a, and Cas12b proteins are commercially available, commonly referred to as dCas9, dCas12a (dCpf1) and dCas12b (dC2c1).

The catalytic domain of the Cas13 protein includes two HEPN domains (higher eukaryotes and prokaryotes nucleotide-binding domain) which confer RNase activity. Examples of mutations that inactivate Cas13 include R474A and R1046A (located at the HEPN domain) for dLwCas13a.

A “proximity tagging enzyme” refers to an enzyme in a proximity tagging system. A proximity tagging system typically includes an enzyme (e.g., Pup ligase, biotin ligase, ascorbate peroxidase) and a substrate (e.g., Pup, biotin, ascorbate). The enzyme can perform the enzymatic reaction on the substrate when the enzyme is in proximity with another required substrate. For instance, a Pup ligase can conjugate a Pup protein to a target protein when the Pup ligase is close to the target protein, thereby tagging the target protein with the Pup protein. Non-limiting examples of proximity tagging systems are provided in the table below.

TABLE C
Example Proximity Tagging Systems

| Proximity | | |
|---|---|---|
| Tagging System | Source | Enzyme activity |
| BioID (BirA*) | E. Coli | Biotin Ligase |
| PUP-IT | Corynebacterium glutamicum | Pup ligase |
| TurboID | E. Coli | Biotin Ligase |
| MiniTurbo | E. Coli | Biotin Ligase |
| BioDI2 | A. Aeolicus | Biotin Ligase |
| BASU | B. Subtilis | Biotin Ligase |
| APEX | Pea (synthetic) | Ascorbate peroxidase |
| APEX2 | Soybean(synthetic) | Ascorbate peroxidase |

In a PUP-IT (Puplyation-based Interacting Tagging) system, the tagging enzyme is a prokaryotic ubiquitin-like protein (Pup) ligase in the Pup bacteria protein-conjugating system, PafA. Pup is a small bacteria protein that carries about 64 amino acids with Gly-Gly-Gln at the C-terminus. When the C-terminus Gln is deaminated to Glu (this form of Pup will be referred to as Pup(E)), in the presence of ATP, Pup ligase PafA can catalyze the phosphorylation of the Pup(E) C-terminus Glu, which in turn conjugates the C-terminus Glu to a lysine residue side chain on the target protein.

“Prokaryotic ubiquitin-like protein” or “Pup” is a functional analog of ubiquitin found in the prokaryote Mycobacterium tuberculosis. It serves the same function as ubiquitin, although the enzymology of ubiquitylation and pupylation is different. In contrast to the three-step reaction of ubiquitylation, pupylation requires two steps, therefore only two enzymes are involved in pupylation. Similar to ubiquitin, Pup attaches to specific lysine residues of substrate proteins by forming isopeptide bonds. It is then recognized by Mycobacterium proteasomal ATPase (Mpa) by a binding-induced folding mechanism that forms a unique alpha-helix. Mpa then delivers the Pup-substrate to the 20S proteasome by coupling of ATP hydrolysis for proteasomal degradation.

There are an abundance of known Pup proteins, which have well reserved amino acid sequences. For instance, a known Pup protein Superfamily (ID: pfam05639) includes 28 Pup proteins. In addition, the table below lists a number of Pup proteins as well as a truncated one (named “Truncated”) which was derived from BAV23336.1 and tested in the experimental examples.

TABLE D
Example Pup Proteins
Example Pup Proteins

| | |
|---|---|
| BAV23336 | MSVVNAK-QTQIM--GG-GGRDEDNTEDSAQASGQVQINTEGVDSLLDEIDGLLENNAEE |
| Truncated | -------------------------------------------DSLLDEIDGLLENNAEE |
| WP_020934768 | ---MTNP-QSQIS--GG-GDRPEDTNDD-AQGLGQAQVNTAGTDDLLDEIDGLLEENAEE |
| WP 066587666 | MTTGGSG-QGQVH--GGRGRGDGPASGD-VTASGQEQLKVSGTDDLLDEIDGLLESNAEE |
| WP 081106290 | ----------MNA--GG-PNADDDSLDH-SLGTAQAQISATGVDDLLDEIDGLLENNAEE |
| WP_006840328 | -----MA-QQQIH--GG-SGNGSEDEGA--FEAGQAQLNTSGTDDLLDETDALLDNNAEE |
| WP 066525612 | -----MSNQQQIH -GH-TGGGDDAEGT-PAQAGQAQINTAGTDDLLDEIDALLDTNAEE |
| WP_003845807 | ---MSNK-QSQVQ--GS-GSGDNSDDDD-VQAAGQVQINTTGTDDLLDEIDGLLESNAEE |
| WP_016457481 | -----MA-DKQVY-SSG-GKGPTDDDVV-DGGAGQVQINTHEADSLLDEIDSLLETDSEE |
| WP_076598554 | -----MA-QDQINISGG-GDNGEGEPGD-ARNAGQVNVNTTGTDDLLDEIDALLDTNAEE |
| BAV23336 | FVRSYVQKGGE (SEQ ID NO: 1) |
| Truncated | FVRSYVQKGGE (SEQ ID NO: 2) |
| WP_020934768 | FVSSYVQKGGQ (SEQ ID NO: 3) |
| WP_066587666 | FVKSYVQKGGQ (SEQ ID NO: 4) |
| WP_081106290 | FVRSYVQKGGQ (SEQ ID NO: 5) |
| WP_006840328 | FVRSYVQKGGE (SEQ ID NO: 6) |
| WP_066525612 | FVRSFVQKGGQ (SEQ ID NO: 7) |
| WP_003845807 | YVSSYVQKGGQ (SEQ ID NO: 8) |
| WP_016457481 | FVKSYVQKGGQ (SEQ ID NO: 9) |
| WP_076598554 | FVRSYVQKGGQ (SEQ ID NO: 10) |

A Pup protein suitable for use with the present technology, therefore, can be any of the Pup proteins disclosed herein, or their truncated forms that includes, e.g., the C-terminal 28 amino acid residues (e.g., SEQ ID NO: 2). In some embodiments, the C-terminal residue can be Glu or modified from another, natural amino acid to Glu.

The fusion protein, in some embodiments, may include one or more nuclear localization sequences (NLS).

A “nuclear localization signal or sequence” (NLS) is an amino acid sequence that tags a protein for import into the cell nucleus by nuclear transport. Typically, this signal consists of one or more short sequences of positively charged lysines or arginines exposed on the protein surface. Different nuclear localized proteins may share the same NLS. An NLS has the opposite function of a nuclear export signal (NES), which targets proteins out of the nucleus. A non-limiting example of NLS is the internal SV40 nuclear localization sequence (iNLS). Some examples are PKKKRKV (SV40 Large T-antigen; SEQ ID NO:20), KRPAATKKAGQAKKKK (nucleoplasmin; SEQ ID NO:21), AVKRPAATKKAGQAKKKKLD (nucleoplasmin; SEQ ID NO:22), MSRRRKANPTKLSENAKKLAKEVEN (EGL-13; SEQ ID NO:23), PAAKRVKLD (c-Myc; SEQ ID NO:24) and KLKIKRPVK (TUS-protein; SEQ ID NO:25).

Suitable Cas proteins, Pup ligase, and Pup proteins can also include biological equivalents of those specifically known or described herein. The term “biological equivalent” of a protein or polypeptide refers to a polypeptide having a certain degree of homology, or sequence identity, with the amino acid sequence of a reference protein or polypeptide. In some aspects, the sequence identity is at least about 70%, 75%, 80%, 85%, 90%, 95%, 98%, or 99%. In some aspects, the equivalent polypeptide or polynucleotide has one, two, three, four or five addition, deletion, substitution and their combinations thereof as compared to the reference protein or polypeptide. In some aspects, the equivalent sequence retains the activity (e.g., RNase, or conjugating to a lysine) or structure of the reference sequence.

In some embodiments, the amino acid substitution is a conservative amino acid substitution. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members.

The term “Pup ligase” or “Pup-protein ligase” refers to a group of proteins which, in the presence of ATP, catalyzes the phosphorylation of the C-terminus Glu of a Pup protein, which in turn conjugates the C-terminus Glu to a lysine residue side chain on a target protein. Pup ligases have well reserved amino acid sequences. Some of the Pup ligases are classified into a GenBank Superfamily (ID: TIGR03686). An example Pup ligase is “Pup-protein ligase [Corynebacterium glutamicum]” (Access No: OKX85684.1), the amino acid sequence of which is listed in the table below.

TABLE E
Pup protein ligase
Sequence of Pup-protein ligase
[Corynebacterium glutamicum]
(SEQ ID NO: 11)

>OKX85684.1 Pup protein ligase
[Corynebacterium glutamicum]
MSTVESALTRRIMGIETEYGLTFVDGDSKKLRPDEIARRMFRPIVEKYSS
SNIFIPNGSRLYLDVGSHPEYATAECDNLTQLINFEKAGDVIADRMAVDA
EESLAKEDIAGQVYLFKNNVDSVGNSYGCHENYLVGRSMPLKALGKRLMP
FLITRQLICGAGRIHHPNPLDKGESFPLGYCVSQRSDHVWEGVSSATTRS
RPIINTRDEPHADSHSYRRLHVIVGDANMAEPSIALKVGSTLLVLEMIEA
DFGLPSLELANDIASIREISRDATGSTLLSLKDGTTMTALQIQQVVFEHA
SKWLEQRPEPEFSGTSNTEMARVLDLWGRMLKAIESGDFSEVDTEIDWVI
KKKLIDRFIQRGNLGLDDPKLAQVDLTYHDIRPGRGLFSVLQSRGMIKRW
TTDEAILAAVDTAPDTTRAHLRGRILKAADTLGVPVTVDWMRHKVNRPEP
QSVELGDPFSAVNSEVDQLIEYMTVHAESYRS

As noted above, once the molecule binds to the protein, the molecule will bring its coupled Pup ligase to the protein. Given that Pup is available in the sample, its C-terminus Glu can be phosphorylated by the Pup ligase which will also conjugate the C-terminus Glu to a lysine residue side chain on the protein.

In some embodiments, the Cas protein is placed at the N-terminal side of the proximity tagging enzyme. In some embodiments, the Cas protein is placed at the C-terminal side of the proximity tagging enzyme. It is demonstrated in the example that such fusion between the Cas protein and the proximity tagging enzyme still allows both of the proteins to be active.

In some embodiments, a linker is placed between the Cas protein and the proximity tagging enzyme. The linker may have a length that is at least 1, 2, 5, 10, 15, 20, 25, 30, 40 or 50 amino acid residues, in some embodiments. In some embodiments, the linker has a length that is not longer than 500, 400, 300, 200, 150, 100, 90, 80, 70, 60, 50, 40, 35, 30, 25, or 20 amino acid residues. In some embodiments, the fusion protein further includes a market protein such as GFP, YFP, and RFP.

### Methods for Detecting Nucleic Acid-Molecule Interactions

The fusion protein can be used to study RNA-molecule interactions. In some embodiments, a method is provided for identifying a molecule that binds to a target nucleic acid. The method may entail contacting a biological sample that includes the target nucleic acid with a fusion protein of the present disclosure, in the presence of a guide RNA that is specific to the target nucleic acid, under conditions to allow the Cas protein to bind to the target nucleic acid and the proximity tagging enzyme to tag molecules bound to the target nucleic acid. Once the molecule is so tagged, it can be isolated and identified.

The proximity tagging enzyme, for instance, can be a Pup ligase, such as PafA. Accordingly then, the contacting is made in the presence of a Pup ligase substrate, PupE. If the proximity tagging enzyme is a biotin ligase, then the contacting can occur in the present of biotin.

The guide RNA can be any that allows the Cas protein to selectively bind to the target nucleic acid. In some embodiments, the guide RNA is a single guide RNA (sgRNA). Methods for designing suitable sgRNA for nucleic acid targeting are well known in the art.

In some embodiments, the contacting is in vitro, in vivo, ex vivo, without limitation. As discussed herein, the present technology allows study of nucleic acid -molecule interactions in their natural state, including in vivo.

### Transgenic Models for Detecting Nucleic Acid-Molecule Interactions

Transgenic organisms can be used for detecting nucleic acid-molecule interactions in the organisms. For instance, Example 2 prepared transgenic mouse and Drosophila models the contained recombinant polynucleotide encoding the fusion protein regulated by an inducible promoter. The fusion protein can be expressed at the desired cells and/or at the desired stage.

The guide RNA, e.g., sgRNA, can be provided either by a recombinant DNA which can be constantly expressed (as no toxicity is expected), induced, or introduced by viral vector (e.g., AVV). Some of the proximity tagging enzymes can required another factor to function. For instance, when the PufA is used as the proximity tagging enzyme, the PupE cDNA can be introduced into the model with an AAV vector.

In one embodiment, therefore, provided is a non-human transgenic organism, comprising a recombinant polynucleotide in at least one cell of the organism, wherein the polynucleotide encodes a fusion protein comprising a clustered regularly interspaced short palindromic repeats (CRISPR)-associated (Cas) protein Cas13 and a proximity tagging enzyme. In some embodiments, the proximity tagging enzyme is selected from the group consisting of a Pup ligase, a biotin ligase, and an ascorbate peroxidase. Examples of proximity tagging enzyme are provided herein.

In a preferred embodiment, the proximity tagging enzyme is PafA. In another preferred embodiment, the proximity tagging enzyme is TurboID or miniTurbo. The PUP-IT system is herein shown as an efficient proximity tagging system for the intended purpose. The TurboID/miniTurbo enzymes, on the other hand, offer the simplicity of not requiring an additional protein for their tagging activities.

In some embodiments, the polynucleotide further comprises an inducible promoter or a tissue-specific promoter that is operably linked and regulates the expression of the fusion protein.

Inducible promoters may be inducible by Cu²⁺, Zn²⁺, tetracycline, tetracycline analog, ecdysone, glucocorticoid, tamoxifen, or an inducer of the lac operon. The promoter may be inducible by ecdysone, glucocorticoid, or tamoxifen. In specific embodiments, the inducible promoter is a phage inducible promoter, nutrient inducible promoter, temperature inducible promoter, radiation inducible promoter, metal inducible promoter, hormone inducible promoter, steroid inducible promoter, or combination thereof. Examples of radiation inducible promoters include fos promoter, jun promoter, or erg promoter. An example of heat inducible promoter is UAS.

A tissue specific promoter can be a liver fatty acid binding (FAB) protein gene promoter, insulin gene promoter, transphyretin promoter, α1-antitrypsin promoter, plasminogen activator inhibitor type 1 (PAI-1) promoter, apolipoprotein AI promoter, LDL receptor gene promoter, myelin basic protein (MBP) gene promoter, glial fibrillary acidic protein (GFAP) gene promoter, opsin promoter, LCK promoter, CD4 promoter, keratin promoter, myoglobulin promoter, or neural-specific enolase (NSE) promoter.

The induction can also be achieved with the Cre-LoxP system, in which the Cre protein can be activated by tamoxifen which then removes the LoxP sequence from the regulated gene.

Methods of using the transgenic organisms are also provided for identifying a protein that binds to a target RNA The method can entail contacting activating the inducible promoter in the non-human transgenic organism in the presence of a guide RNA that is specific to the target RNA, under conditions to allow the Cas13 protein to bind to the target RNA and the proximity tagging enzyme to tag proteins bound to the target RNA.

The guide RNA may be introduced with a viral vector such as AAV, or expressed from a recombinant polynucleotide in the non-human transgenic organism, without limitation.

### Fusion Proteins, Conjugates, Compositions and Kits

Fusion proteins, conjugates, compositions and kits are also provided which are useful for carrying out certain embodiments of the present technology.

In some embodiments, a kit or package is provided comprising a fusion protein of the present disclosure and a substrate for the proximity tagging enzyme to tag a molecule with. In some embodiments, the proximity tagging enzyme is PafA and the substrate is a Pup protein. In some embodiments, the kit or package further include a suitable guide RNA.

Polynucleotides are also provided that encode any of the proteins disclosed herein. In some embodiments, cells are provided that contain a polynucleotide or protein of the present disclosure.

### EXAMPLES

### Example 1

### Capturing RNA-Protein Interaction

This example demonstrates the development of a new tool, CRISPR-based RNA-United Interacting System (CRUIS), which uses the CRISPR-based RNA-target Cas nuclease as an RNA tracker to bring the proximity-labeling system to a designated target RNA. CRUIS can capture RNA-protein interactions of specific RNA sequences effectively. In CRUIS, a dead RNA-guided RNA targeting nuclease LwaCas13a (dLwaCas13a) was used as a tracker to target specific RNA sequences, while proximity enzyme PafA was fused to dLwaCas13a to label surrounding RNA-binding proteins. Subsequently, the labeled proteins were enriched and identified by mass spectrometry.

### Methods and Materials

### Cell Culture and Generation of Stable Cell Line

HEK293T cells were grown in DMEM (Hyclone) supplemented with 10% FBS (Biological Industries) in a humidified incubator at 37° C. with 5% CO₂. All constructs were prepared using E.Z.N.A.® Endo-free Plasmid DNA Mini Kit (Omega, cat. #D6950-01B) and transfected with Lipofectamine 2000 (Thermo, cat. #11668019). The sequence of CRUIS is available in Table 1. Stable cell lines were generated with the piggyBac transposon system, which is widely applicable to various cell lines including non-mammalian cell lines. GFP-positive cells were enriched by flow sorting after transfection. Single colonies were picked, expanded, and tested via PCR, western blot, and enzyme activity identification for PafA. The HEK293T cell line with the best inducibility (referred to as 293T-CRUIS) was expanded and used for all subsequent experiments.

TABLE 1
Amino acid sequence of dLwaCas13a-PafAP2A-
EGFP fusion protein
Amino acid sequence of dLwaCas13a-PafA-
P2A-EGFP (SEQ ID NO: 12)

| | |
|---|---|
| 1 | MPKKKRKVGR VCRISSLRYR GPGIATMKVT KVDGISHKKY |
| | IEEGKLVKST |
| 51 | SEENRTSERL SELLSIRLDI YIKNPDNASE EENRIRRENL |
| | KKFFSNKVLH |
| 101 | LKDSVLYLKN RKEKNAVQDK NYSEEDISEY DLKNKNSFSV |
| | LKKILLNEDV |
| 151 | NSEELEIFRK DVEAKLNKIN SLKYSFEENK ANYQKINENN |
| | VEKVGGKSKR |
| 201 | NIIYDYYRES AKRNDYINNV QEAFDKLYKK EDIEKLFFLI |
| | ENSKKHEKYK |
| 251 | IREYYHKIIG RKNDKENFAK IIYEEIQNVN NIKELIEKIP |
| | DMSELKKSQV |
| 301 | FYKYYLDKEE LNDKNIKYAE CHFVEIEMSQ LLKNYVYKRL |
| | SNISNDKIKR |
| 351 | IFEYQNLKKL IENKLLNKLD TYVRNCGKYN YYLQVGEIAT |
| | SDFIARNRQN |
| 401 | EAFLRNIIGV SSVAYFSLRN ILETENENDI TGRMRGKIVK |
| | NNKGEEKYVS |
| 451 | GEVDKIYNEN KQNEVKENLK MFYSYDFNMD NKNEIEDFFA |
| | NIDEAISSIA |
| 501 | HGIVHFNLEL EGKDIFAFKN IAPSEISKKM FQNEINEKKL |
| | KLKIFKQLNS |
| 551 | ANVFNYYEKD VIIKYLKNTK FNFVNKNIPF VPSFTKLYNK |
| | IEDLRNTLKF |
| 601 | FWSVPKDKEE KDAQIYLLKN IYYGEFLNKF VKNSKVFFKI |
| | TNEVIKINKQ |
| 651 | RNQKTGHYKY QKFENIEKTV PVEYLAIIQS REMINNQDKE |
| | EKNTYIDFIQ |
| 701 | QIFLKGFIDY LNKNNLKYIE SNNNNDNNDI FSKIKIKKDN |
| | KEKYDKILKN |
| 751 | YEKHNRNKEI PHEINEFVRE IKLGKILKYT ENLNMFYLIL |
| | KLLNHKELIN |
| 801 | LKGSLEKYQS ANKEETFSDE LELINLLNLD NNRVTEDFEL |
| | EANEIGKFLD |
| 851 | FNENKIKDRK ELKKFDINKI YFDGENIIKH RAFYNIKKYG |
| | MLNLLEKIAD |
| 901 | KAKYKISLKE LKEYSNKKNE TEKNYTMQQN LHRKYARPKK |
| | DEKFNDEDYK |
| 951 | EYEKAIGNIQ KYTHLKNKVE FNELNLLQCL LLKILHRLVG |
| | YTSIWERDLR |
| 1001 | FRLKGEFPEN HYIEEIFNFD NSKNVKYKSG QIVEKYINFY |
| | KELYKDNVEK |
| 1051 | RSIYSDKKVK KLKQEKKDLY IANYIAHFNY IPHAEISLLE |
| | VLENLRKLLS |
| 1101 | YDRKLKNAIM KSIVDILKEY GFVATFKIGA DKKIEIQTLE |
| | SEKIVHLENL |
| 1151 | KKKKLMTDRN SEELCELVKV MFEYKALEQR PQGGGGPKKK |
| | RKVGGSGMST |
| 1201 | VESALTRRIM GIETEYCLTE VDGDSKKLRP DEIARRMFRP |
| | IVEKYSSSNI |
| 1251 | FIPNGSRLYL DVGSHPEYAT AECDNLTQLI NFEKAGDVIA |
| | DRMAVDAEES |
| 1301 | LAKEDIAGQV YLFKNNVDSV GNSYGCHENY LVGRSMPLKA |
| | LGKRLMPFLI |
| 1351 | TRQLICGAGR THHPNPLDKG ESFPLGYCIS QRSDHVWEGV |
| | SSATTRSRPI |
| 1401 | INTRDEPHAD SHSYRRLHVI VGDANMAEPS IALKVGSTLL |
| | VLEMIEADFG |
| 1451 | LPSLELANDI ASIREISRDA TGSTLLSLKD GTTMTALQIQ |
| | QVVFEHASKW |
| 1501 | LEQRPEPEFS GTSNTEMARV LDLWGRMLKA IESGDFSEVD |
| | TEIDWVIKKK |
| 1551 | LIDRFIQRGN LGLDDPKLAQ VDLTYHDIRP GRGLFSVLQS |
| | RGMIKRWTTD |
| 1601 | EAILAAVDTA PDTTRAHLRG RILKAADTLG VPVTVDWMRH |
| | KVNRPEPQSV |
| 1651 | ELGDPFSAVN SEVDQLIEYM TVHAESYRSE QKLISEEDLG |
| | SGATNFSLLK |
| 1701 | QAGDVEENPG PMVSKGEELF TGVVPILVEL DGDVNGHKFS |
| | VSGEGEGDAT |
| 1751 | YGKLTLKFIC TTGKLPVPWP TLVTTLTYGV QCFSRYPDHM |
| | KQHDFFKSAM |
| 1801 | PEGYVQERTI FFKDDGNYKT RAEVKFEGDT LVNRIELKGI |
| | DFKEDGNILG |
| 1851 | HKLEYNYNSH NVYIMADKQK NGIKVNFKIR HNIEDGSVQL |
| | ADHYQQNTPI |
| 1901 | GDGPVLLPDN HYLSTQSALS KDPNEKRDHM VLLEFVTAAG |
| | ITLGMDELYK |

### Plasmid Construction

The CRUIS construct (dLwaCas13a-PafA-P2A-EGFP) was generated by subcloning dLwaCas13a fused with PafA at the C-terminus and a self-cleaving P2A peptide-linked EGFP (enhanced green fluorescent protein) into a piggyBac transposon backbone. dLwaCas13a was obtained by introducing two point mutations (R474A and R1046A) in the LwaCas13a (Addgene plasmid #90097) HEPN domains. The PafA was obtained from pEF6a-CD28-PafA (Addgene plasmid #113400). ClonExpress MultiS One Step Cloning Kit (Vazyme, cat. #C113-01) and Mut Express II Fast Mutagenesis Kit V2 (Vazyme, cat. #C214-01) were used for construct generation. The CRUIS plasmid will be deposited to the open-access platform Addgene.

### Tracking Stress Granules by CRUIS

293T-CRUIS cells were plated in 24-well tissue culture plates on poly-d-lysine coverslips and transfected with 500 ng ACTB-sgRNA, and then 100 mM sodium malonate was applied for 1.5 h before fixing and permeabilizing the cells. For immunofluorescence of G3BP1, cells were blocked with 5% BSA and incubated overnight at 4° C. with anti-G3BP1 primary antibody (Proteintech, cat. #13057-2-AP), and anti-myc primary antibody (Cell Signaling, cat. #9B11). Cells were then incubated for 2 h at room temperature with secondary antibody and mounted using the anti-fade mounting medium.

### RNA Extraction and Quantitative Real-Time PCR

Total RNAs from 5×10⁵ 293T cells were extracted with Trizol (Invitrogen, Cat. #15596026) and RNA concentration were determined by NanoDrop 2000c (Thermo Fisher). cDNA was synthesized using 1 μg RNA by the reverse transcription kit PrimeScript™ II 1st Strand cDNA Synthesis Kit (TaKaRa, Cat. #6210A) according to the manufacturer's instructions. Each qRT-PCR reaction was performed with cDNA transcribed from 25 ng RNA in a final volume of 20 μl with ChamQ™ SYBR Color qPCR Master Mix (Vazyme Cat. #Q431-03), assayed by QuantStudio™ 7 Flex (Life Technologies). The qPCR data were normalized to GAPDH expressions by relative quantification (ΔΔCt) method. The primers used were: CXCR4 (forward primer, 5′-ACTACACCGAGGAAATGGGCT-3′, SEQ ID NO:26; reverse primer, 5′-CCCACAATGCCAGTTAAGAAGA-3′, SEQ ID NO:27), p21 (forward primer, 5′-TGTCCGTCAGAACCCATGC-3′, SEQ ID NO:28; reverse primer, 5′-AAAGTCGAAGTTCCATCGCTC-3′, SEQ ID NO:29); NORAD (forward primer, 5′-CAGAGGAGGTATGCAGGGAG-3′, SEQ ID NO:30; reverse primer, 5′-GGATGTCTAGCTCCAAGGGG-3′, SEQ ID NO:31), β-actin (forward primer, 5′-CATGTACGTTGCTATCCAGGC-3′, SEQ ID NO:32; reverse primer, 5′-CTCCTTAATGTCACGCACGAT-3′, SEQ ID NO:33). GAPDH (forward primer, 5′-AGATCCCTCCAAAATCAAGTGG-3′, SEQ ID NO:34; reverse primer, 5′-GGCAGAGATGATGACCCTTTT-3′, SEQ ID NO:35).

### Western Blot

293T-CRUIS cell lines transfected with or without pCMV-bio-pupE were analyzed by western blot. About 2 million cells were harvested and washed with cold PBS. Lysis buffer (1% Triton, 50 mM Tris 7.5, 150 mM NaCl) with 100× protease inhibitor was added to the pellet. Cells were resuspended and incubated on ice for 1 h. Then the lysate was spun down and the supernatant collected with the addition of protein loading buffer. The samples were boiled at 100° C. for 10 min and loaded on 4-20% SDS-PAGE gels, followed by immune-bolting with anti-myc antibody and streptavidin-HRP (Cell Signaling, cat. #3999s) to identify the expression of dCas13a-PafA fusion protein and the activity of PafA ligase.

For the enrichment of Bio-PupE modified proteins by streptavidin magnetic beads. Thirty-six hours after transfection with sgRNA or non-target sgRNA into the 293T-CRUIS cell line, the treated cells were harvested and lysed using cell lysate buffer. 20 μl streptavidin magnetic beads used for capturing labeled proteins from cell lysate supernatant and washed 3 times by wash buffer (8 M urea, 50 mM Tris 8.0, 200 mM NaCl). The obtained proteins were boiled at 100° C. for 20 min and used for western blot to analyze whether HNRNPK was modified by Bio-PupE, HNRNPK was identified by specific antibody (Proteintech, cat. #11426-1-AP).

### Mass Spectrometry Preparation

About 30 million cells transfected with pCMV-bio-pupE and sgRNA were used for the mass spectrum. Cells were harvested and washed with cold PBS, then incubated with 2 ml lysis buffer at 4° C. After shaking for 1 h, the lysate was spun down at 4° C. for 10 min The supernatant was transferred into new tubes, with the addition of urea and DTT to a final concentration of 8 M and 10 mM. The lysate was incubated at 56° C. for 1 hour, then treated with 25 mM iodoacetamide in the dark for 45 min to aminocarbonyl modify the Cys site of proteins. 25 mM DTT was added to terminate the modification. Streptavidin-biotin magnetic beads were washed with 500 μl PBS three times and then resuspended in lysis buffer with an equal volume of beads. The lysate was then added 50 μl beads and it was incubated on a rotator at 4° C. overnight. The beads were washed with the following buffers: twice with buffer 1 (50 mM Tris 8.0, 8 M urea, 200 mM NaCl, 0.2% SDS), once with buffer 2 (50 mM Tris 8.0, 200 mM NaCl, 8 M urea), twice with buffer 3 (50 mM Tris 8.0, 0.5 mM EDTA, 1 mM DTT), three times with buffer 4 (100 mM ammonium carboxylate), and finally the beads were resuspended in 100 μl buffer 4. Trypsin, 4 μg (Promega, cat. #v5113) was added to digest overnight at 37° C. The peptides were collected with ziptip by the addition of 1% formic acid, then washed with 0.1% TFA (Sigmal, cat. #14264) and eluted in 50 μl of 70% ACN (Merck Chemicals, cat. #100030) −0.1% TFA. The peptides were analyzed on an Orbitrap Fusion.

### Mass Spectrometry Data Analysis

For statistical analysis, the R package Limma was applied for the analysis of LFQ intensity data. The target RNA binding proteins were determined by a moderated t-test (p. value<0.05) and fold change (fold change>3). Previously reported RNA binding proteins were obtained from StarBase v2.0 (starbase.sysu.edu.cn). The R package clusterProfiler was used to identify significantly enriched biological processes in the RNA interactome (p-value cutoff=0.01, q-value cutoff=0.05, p. adjust method=Benjamini & Hochberg). The subcellular localization of the identified RBPs was analyzed by an online gene annotation & analysis resource “Metascape” (www.metascape.org). All data visualization was implemented in R using the ggplot2 package.

### RNA Immunoprecipitation

For RNA immunoprecipitation experiments, HEK293T cells were plated in a 6-cm dish and transfected with target protein expression plasmid (labeled with HA-tag at the C-terminus). Thirty-six hours after transfection, proteins were crosslinked to RNA by adding formaldehyde drop-wise directly to the medium to a final concentration of 0.75% and rotating gently at room temperature for 15 min. After crosslinking, 125 mM glycine in PBS was used for quenching, and the cells were incubated for 10 min at room temperature. Cells were washed with ice-cold PBS, harvested by scraping, and the cell suspension was centrifuged at 800 g for 4 min to pellet the cells. Cells were lysed with RIPA buffer supplemented with Protease Inhibitor Cocktail, EDTA-free and Recombinant RNasin® Ribonuclease Inhibitor (Promega cat. #N2515). Cells were allowed to lyse on a rotator for 20 min at 4° C. and then sonicated for 2 min with a 30 s on/30 s off cycle at low intensity on a Bioruptor sonicator (Diagenode) at 4° C. Insoluble material was pelleted by centrifugation at 16,000 g for 10 min at 4° C., and the supernatant containing the clarified lysate was split into two portions for pulling down with anti-HA magnetic beads (bimake cat. #B26202) or mouse IgG-conjugated magnetic beads overnight in a rotator at 4° C. After incubation with sample lysate, beads were pelleted, washed three times with RIPA buffer, and then washed with 1×DNase buffer (RNase-free). Beads were resuspended in 100 μl DNase buffer (RNase-free). DNase I (RNase-free) was added, followed by incubation at 37° C. for 30 min on a rotator. Proteins were then digested by the addition of Proteinase K (Takara cat. #9034) for about 2 hours at 37° C. with rotation. After that, MicroElute RNA Clean Up Kit (Omega cat. #R6247-01) was used for RNA purification. Purified RNA was reverse transcribed to cDNA using PrimeScript™ II 1st Strand cDNA Synthesis Kit (TaKaRa, cat. #6210A), and pulldown was quantified with qPCR using ChamQ™ SYBR Color qPCR Master Mix (Vazyme cat. #Q431-03) and the Life Technologies QuantStudio™ 7 Flex. Enrichment was quantified for samples compared with their matched IgG antibody controls. The primers used for RIP-qPCR were: forward primer, 5′-GACAGGCCGAGCCCTCTGC-3′; reverse primer, 5′-GGCTTCAAGGTCTGGGCACAGC-3′.

### Results

### Development of CRUIS

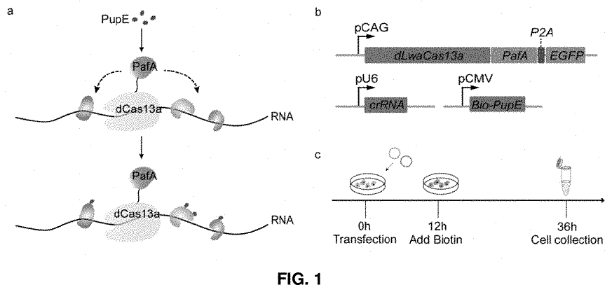

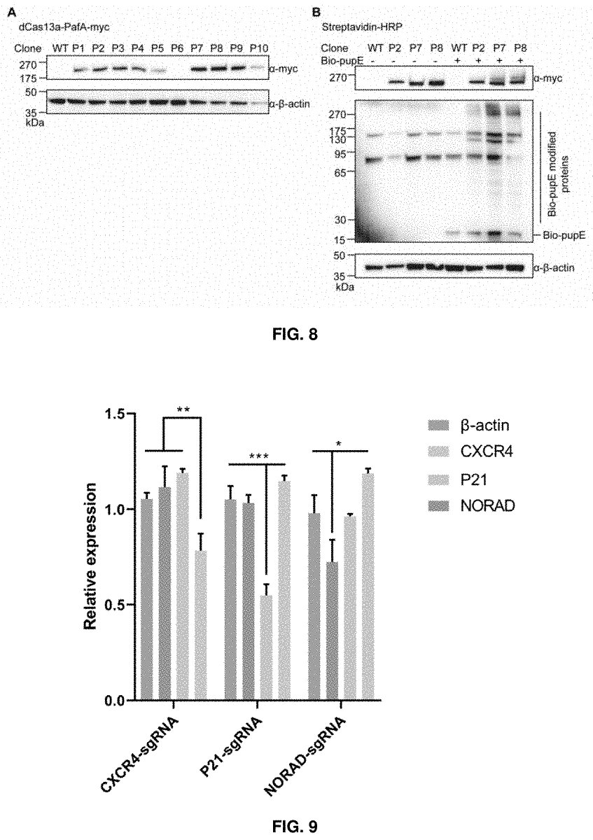

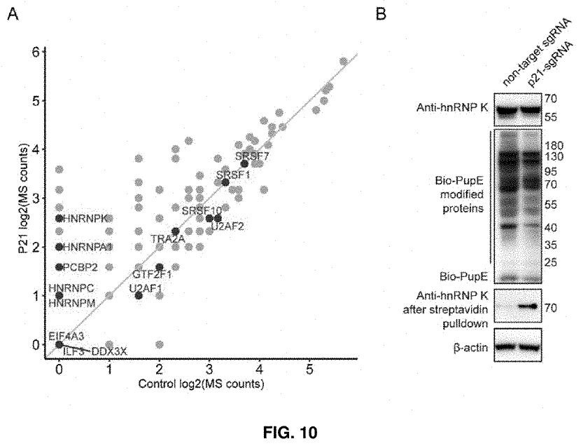

To implement CRUIS in cells, this example first constructed a transfection vector which fused dLwaCas13a and PafA, and then cloned the fused dLwaCas13a-PafA gene in-frame with the self-cleaving P2A peptide sequence and EGFP, and the fusion gene driven by a CAG promoter (FIG. 6, Table 1). In addition, because PafA has a cytoplasmic tendency, in order to enable CRUIS to be widely applied to RNA distributed in the nucleus and cytoplasm, this example introduced NLS sequences (FIGS. 1B and 6). Using EGFP this example observed that the introduction of NLS does not result in the complete distribution of CRUIS in the nucleus due to PafA, but in the nucleus and cytoplasm, which confers versatility (FIG. 7).

In order to express dLwaCas13a-PafA at certain levels, this example created a monoclonal HEK293T cell line with stably integrated dLwaCas13a-PafA (referred to as 293T-CRUIS) by the piggyBac transposon system. For 293T-CRUIS cells, it is only necessary to transfect an expression vector of sgRNA and PupE to achieve the labeling of the RNA-binding proteins of target RNAs (FIG. 1C). The obtained monoclonal cell line was to be used for further testing, including whether the dLwaCas13a-PafA fusion protein had proximity targeting activity and whether it could bind to the target RNA.

### Detection of Proximity Targeting Activity

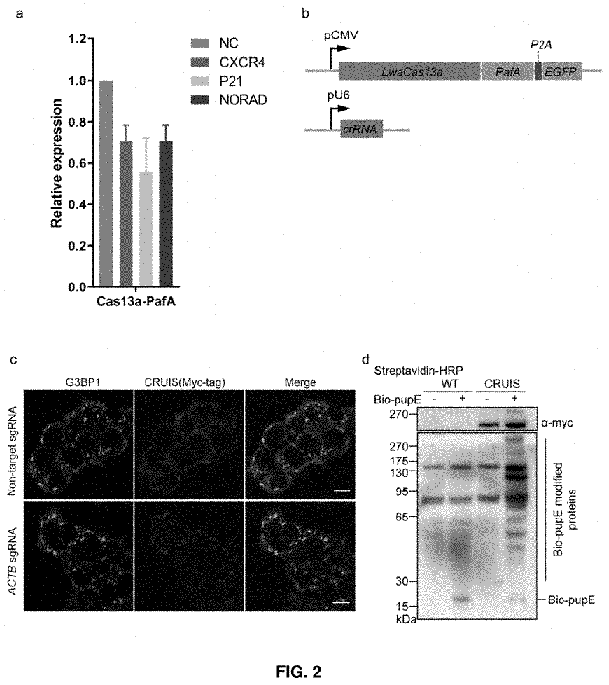

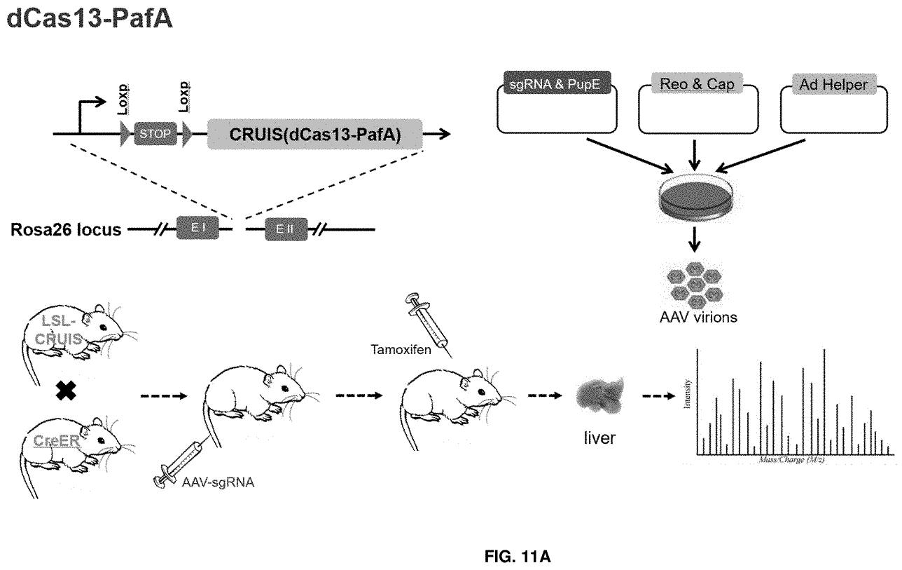

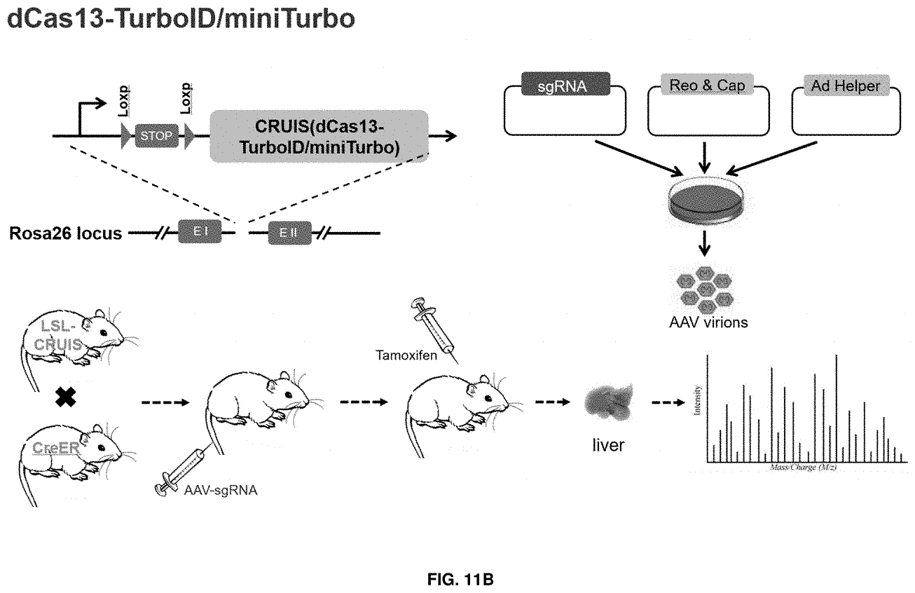

To determine whether CRUIS can bind to the target RNA, retain normal catalytic activity, and label surrounding proteins, this example first selected several 293T-CRUIS cell lines and determined the proximity targeting activity. It was confirmed that PafA retained the ability to label adjacent proteins in 293T-CRUIS cells (FIG. 8). In addition, this example investigated whether CRUIS could bind to the target RNA. Since binding to the target RNA is a prerequisite for clearance, this example first examined whether LwaCas13a-PafA could knock down the expression level of the target RNA. As expected, LwaCas13a-PafA performed well in knocking down target RNA (FIGS. 2A, 2B, and 9, and Table 2).

TABLE 2
Biological processes information

| ID | Description |
|---|---|
| GO:0006397 | mRNA processing |
| GO:0008380 | RNA splicing |
| GO:0000375 | RNA splicing, via transesterification reactions |
| GO:0000377 | RNA splicing, via transesterification reactions |
| | with bulged adenosine as nucleophile |
| GO:0000398 | mRNA splicing, via spliceosome |
| GO:1903311 | regulation of mRNA metabolic process |
| GO:0006403 | RNA localization |
| GO:0050657 | nucleic acid transport |
| GO:0050658 | RNA transport |
| GO:0051236 | establishment of RNA localization |
| GO:0015931 | nucleobase-containing compound transport |
| GO:0043484 | regulation of RNA splicing |
| GO:0050684 | regulation of mRNA processing |
| GO:0048024 | regulation of mRNA splicing, via spliceosome |
| GO:1903312 | negative regulation of mRNA metabolic process |
| GO:0031124 | mRNA 3′-end processing |
| GO:0031123 | RNA 3′-end processing |
| GO:0050685 | positive regulation of mRNA processing |
| GO:1903313 | positive regulation of mRNA metabolic process |
| GO:0033120 | positive regulation of RNA splicing |
| GO:0006614 | SRP-dependent cotranslational protein targeting |
| | to membrane |
| GO:0006613 | cotranslational protein targeting to membrane |
| GO:0045047 | protein targeting to ER |
| GO:0072599 | establishment of protein localization to |
| | endoplasmic reticulum |
| GO:0000184 | nuclear-transcribed mRNA catabolic process, |
| | nonsense-mediated decay |
| GO:0070972 | protein localization to endoplasmic reticulum |
| GO:0006612 | protein targeting to membrane |
| GO:0019083 | viral transcription |
| GO:0006413 | translational initiation |
| GO:0019080 | viral gene expression |
| GO:0000956 | nuclear-transcribed mRNA catabolic process |
| GO:0090150 | establishment of protein localization to membrane |
| GO:0006402 | mRNA catabolic process |
| GO:0006401 | RNA catabolic process |
| GO:0072594 | establishment of protein localization to organelle |

To further confirm whether CRUIS would be able to recognize target RNA with a specific sgRNA, this example used ACTB-targeted sgRNA to determine whether CRUIS colocalizes with ACTB-containing stress granules under conditions induced by sodium malonate. Twenty-four hours after transfecting ACTB-targeting sgRNA into the 293T-CRUIS cell line, stress granules were induced by adding 100 mM sodium malonate into the culture medium Immunochemical labeling with an antibody against the stress granule marker G3BP1 demonstrated that CRUIS had been recruited specifically into the stress granules (FIG. 2C).

### Capturing RBPs of NORAD

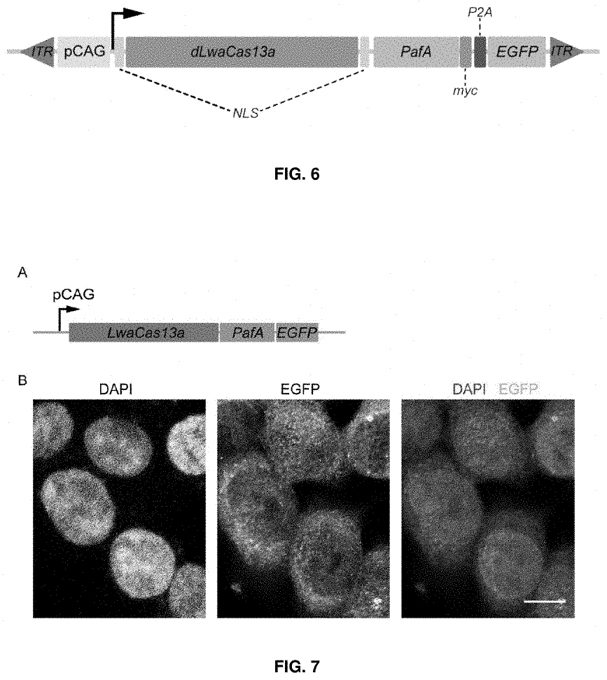

To prove the concept, this example applied CRUIS to study the RBPs of NORAD, a long non-coding RNA. NORAD plays an important role in genomic stability. Moreover, previous studies have suggested that RBPs are critical for the function of NORAD. To this end, this example transfected the NORAD-target sgRNA into the 293T-CRUIS. Biotin was added to the medium at 12 hours after the transfection. Twenty-four hours later, the cells were collected and lysed (FIG. 1C) Then, all biotinylated proteins were pulled down using streptavidin beads. Finally, LC-MS/MS was used to identify the proteins enriched by affinity-based purification (FIG. 5).

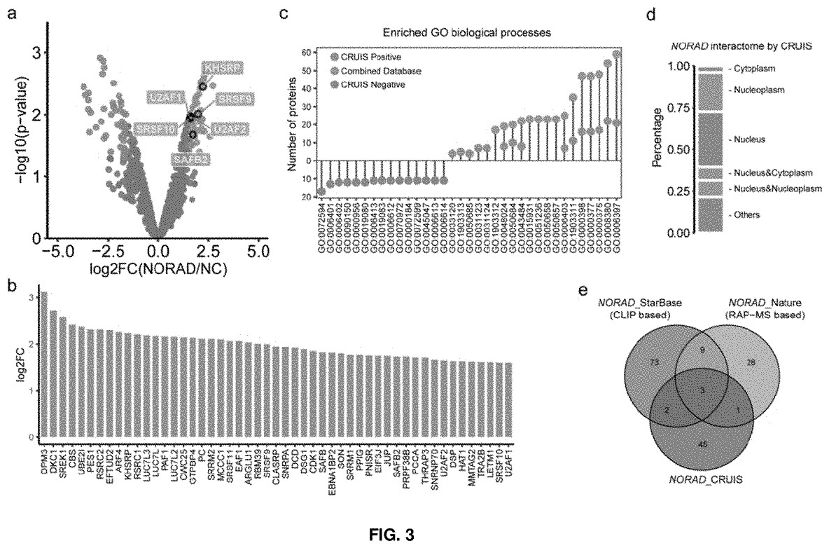

It was found that 51 candidates were significantly enriched in the NORAD targeting sgRNA group (p value<0.05) compared with the non-targeting sgRNA control group (FIG. 3A). Among those 51 candidate proteins, six (KHSRP, SRSF9, U2AF2, SRSF10, U2UF1 and SAFB2) are previously reported NORAD binding proteins. The enrichment of each protein, reflected by the fold changes, is also ranked (FIG. 3B). The top hits include DKC1, SREK1, and RSRC2, which are known RNA binding proteins that play important roles in regulating RNA splicing and mRNA processing.

The candidate NORAD-binding proteins identified by CRUIS are involved in biological processes that are distinct from those of the control sample (FIG. 3C, Table 3). The top biological processes characterized as related to the function of NORAD binding proteins are RNA splicing (GO:0008380), mRNA processing (GO:0006397), and RNA splicing via transesterification reactions (GO:0000375). Furthermore, the subcellular localization analysis of the identified NORAD-binding proteins also shows a significant enrichment of nuclear proteins (FIG. 3D).

TABLE 3
sgRNA information

| Name | Guide sequence (5′-3′) | FIGS. |
|---|---|---|
| ACTB- | ctggcggcgggtgtggacgggcggcgga | 1C |
| sgRNA | (SEQ ID NO: 36) | |
| NORAD- | tcggcaacctctttccatctagaagggc | 2A, 3A. 9 |
| sgRNA | (SEQ ID NO: 37) | |
| CXCR4- | atgataatgcaatagcaggacaggatga | 2A and 9 |
| sgRNA | (SEQ ID NO: 38) | |
| P21- | tacactaagcacttcagtgcctccaggg | 2A, 9 and 10 |
| sgRNA | (SEQ ID NO: 39) | |

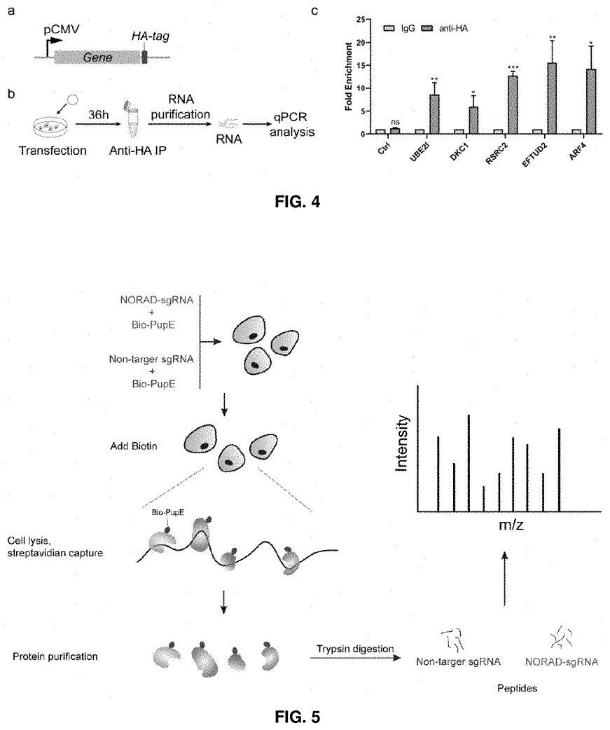

Using CRUIS, this example verified some NORAD-binding proteins identified previously (FIG. 3E). Furthermore, this example performed RIP-qPCR to confirm the several new binding proteins of NORAD from the enriched proteins (FIG. 4A-C).

Capturing RBPs of p21 mRNA

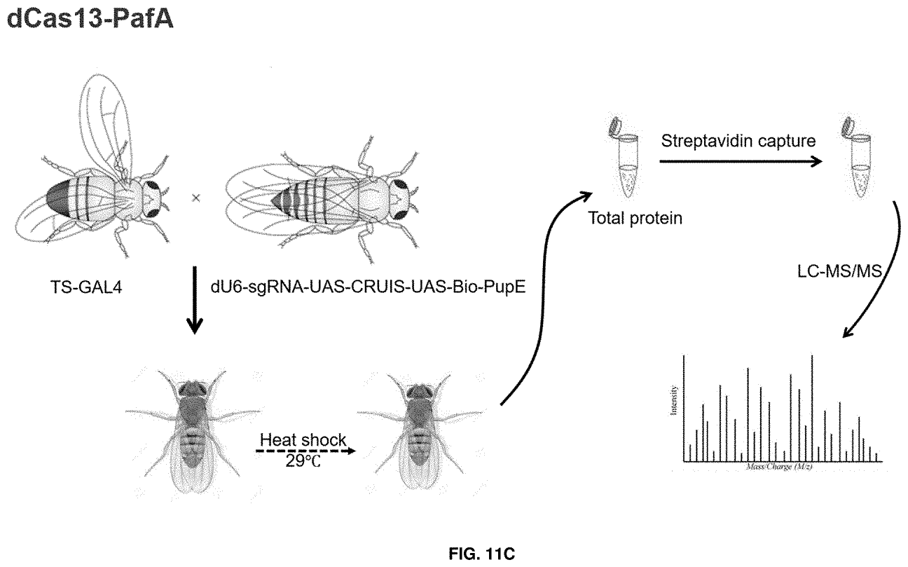

To determine whether CRUIS is able to identify RBPs for mRNAs, this example designed sgRNAs to target p21 mRNA and applied CRUIS. The data from mass spectrometry retrieved putative RBPs for p21 mRNA, some of them are known RBPs of p21 mRNA (marked in red) (FIG. 10A). It was verified that CRUIS can mediate Bio-PupE modification on an RNA-binding protein associating with p21 mRNA (FIG. 10B). The enriched proteins of p21 mRNA are different from the RBPs of NORAD captured by CRUIS. Some of the proteins enriched in the p21-target group, such as HNRNPK, HNRNPA1, HNRNPC and PCBP2, are common proteins that bind most nascent hnRNA. It reflects the different post-transcriptional maturation mechanism between mRNA and long non-coding RNA.

### Example 2

### Mouse/Fruit Fly Models

This example tested transgenic mouse and Drosophila models useful for implementing the CRUIS technology.

dCas13-PafA in Mouse

A construct was prepared that included CRUIS (dCas13-PafA) with LoxP sequences: pCAG-loxp-STOP-loxp-CRUIS. A transgenic mouse was obtained that had the construct integrated at the Rosa26 locus, and through mating with a mouse with CreER.

To activate the CRUIS, an AAV carry a polynucleotide encoding a sgRNA and PupE was injected to the tail of the mouse. The sgRNA and PupE were expressed in the liver of the mouse.

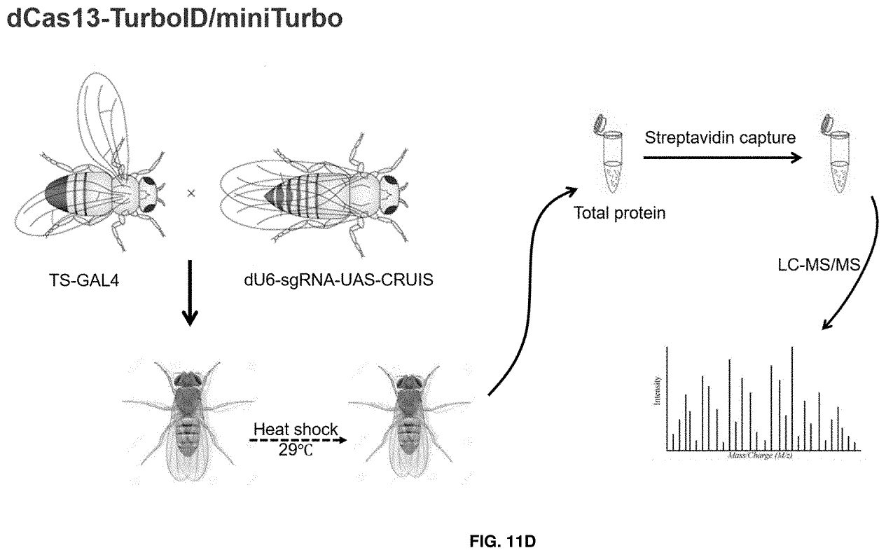

Expression of the CRUIS was triggered by injection of Tamoxifen. After the tagging, additional biotin was supplied with food. The mouse was sacrificed and liver obtained for mass spectrum analysis of the tagged proteins. This process is illustrated in FIG. 11A.

dCas13-PafA in Drosophila

The Drosophila model was prepared similar to the mouse model (see illustration in FIG. 11B). The transgene construct included dU6-sgRNA-UAS-CRUIS-UAS-PupE. The expression of the sgRNA was under the dU6 promoter and the expression of the CRUIS and PupE fusion was under the UAS promoter. The expression of the CRUIS and PupE was activated by heat.

dCas13-TurboID/miniTurbo in Mouse

In this example, the CRUIS used TurboID and miniTurbo as the proximity tagging enzyme. The construct for expression in the mouse (with CreER) included pCAG-loxp-STOP-loxp-CRUIS. The sgRNA was also introduced through an AAV vector, and the expression of the CRUIS was triggered by injected Tamoxifen. This process is illustrated in FIG. 11C.

dCas13-TurboID/miniTurbo in Drosophila

The construct was dU6-sgRNA-UAS-CRUIS, and the process is similar to the Drosophila model above (illustrated in FIG. 11D).

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

What is claimed is:

1. A non-human transgenic organism, comprising a recombinant polynucleotide in at least one cell of the organism, wherein the polynucleotide encodes a fusion protein comprising a clustered regularly interspaced short palindromic repeats (CRISPR)-associated (Cas) protein Cas13 and a proximity tagging enzyme.

2. The non-human transgenic organism of claim 1, wherein the proximity tagging enzyme is selected from the group consisting of a Pup ligase, a biotin ligase, and an ascorbate peroxidase.

3. The non-human transgenic organism of claim 2, wherein the proximity tagging enzyme is PafA.

4. The non-human transgenic organism of claim 2, wherein the proximity tagging enzyme is TurboID or miniTurbo.

5. The non-human transgenic organism of claim 1, wherein the Cas13 is selected from the group consisting of Cas13a, Cas13b, Cas13c, and Cas13d.

6. The non-human transgenic organism of claim 1, wherein the Cas13 is selected from the group consisting of LshCas13a, LwaCas13a, LseCas13a, LbmCas13a, LbnCas13a, CamCas13a, CgaCas13a, Cga2Cas13a, Pprcas13a, LweCas13a, LbfCas13a, Lwa2cas13a, RcsCas13a, RcrCas13a, RcdCas13a, LbuCas13a, HheCas13a, EreCas13a, EbaCas13a, BmaCas13a, LspCas13a, BzoCas13b, PinCas13b, PbuCas13b, AspCas13b, PsmCas13b, RanCas13b, PauCas13b, PsaCas13b, Pin2Cas13b, CcaCas13b, PguCas13b, PspCas13b, FbrCas13b, PgiCas13b, Pin3Cas13b, FnsCas13c, FndCas13c, FnbCas13c, FnfCas13c, FpeCas13c, FulCas13c, AspCas13c, UrCas13d, RffCas13d, RaCas13d, AdmCas13d, PIE0Cas13d, EsCas13d, and RfxCas13d.

7. The non-human transgenic organism of claim 5, wherein the Cas13 is catalytically dead.

8. The non-human transgenic organism of claim 7, wherein the Cas13 is dLwCas13a with an R474A or R1046A mutation.

9. The non-human transgenic organism of claim 1, wherein the polynucleotide further comprises an inducible promoter or a tissue-specific promoter that is operably linked to and regulates the expression of the fusion protein.

10. The non-human transgenic organism of claim 9, wherein the inducible promoter is LoxP or UAS.

11. A method of identifying a protein that binds to a target RNA, comprising contacting activating the inducible promoter in the non-human transgenic organism of claim 9 in the presence of a guide RNA that is specific to the target RNA, under conditions to allow the Cas13 protein to bind to the target RNA and the proximity tagging enzyme to tag proteins bound to the target RNA.

12. The method of claim 11, wherein the guide RNA is introduced with a viral vector.

13. The method of claim 11, wherein the guide RNA is expressed from a recombinant polynucleotide in the non-human transgenic organism.

14. A fusion protein comprising a clustered regularly interspaced short palindromic repeats (CRISPR)-associated (Cas) protein Cas13 and a proximity tagging enzyme.

15. The fusion protein of claim 14, wherein the Cas13 is selected from the group consisting of Cas13a, Cas13b, Cas13c, and Cas13d.

16. The fusion protein of claim 15, wherein the Cas13 is selected from the group consisting of LshCas13a, LwaCas13a, LseCas13a, LbmCas13a, LbnCas13a, CamCas13a, CgaCas13a, Cga2Cas13a, Pprcas13a, LweCas13a, LbfCas13a, Lwa2cas13a, RcsCas13a, RcrCas13a, RcdCas13a, LbuCas13a, HheCas13a, EreCas13a, EbaCas13a, BmaCas13a, LspCas13a, BzoCas13b, PinCas13b, PbuCas13b, AspCas13b, PsmCas13b, RanCas13b, PauCas13b, PsaCas13b, Pin2Cas13b, CcaCas13b, PguCas13b, PspCas13b, FbrCas13b, PgiCas13b, Pin3Cas13b, FnsCas13c, FndCas13c, FnbCas13c, FnfCas13c, FpeCas13c, FulCas13c, AspCas13c, UrCas13d, RffCas13d, RaCas13d, AdmCas13d, PIE0Cas13d, EsCas13d, and RfxCas13d.

17. The fusion protein of claim 14, wherein the Cas13 is catalytically dead.

18. The fusion protein of claim 17, wherein the Cas13 is dLwCas13a with an R474A or R1046A mutation.

19. The fusion protein of claim 14, wherein the proximity tagging enzyme is selected from the group consisting of a Pup ligase, a biotin ligase, and an ascorbate peroxidase.

20. The fusion protein of claim 14, further comprising one or more nuclear localization sequence (NLS).
